SEQUENCE LISTING (5 sequences)

SEQ ID NO: 1 - protein, 601 aa, Artificial sequence
  Acetylcholinesterase readthrough isoform (oAChE-R) polypeptide sequence
MGRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATGMQGPAGSGWEEGSGSPPGVTPLFSP

SEQ ID NO: 2 - protein, 96 aa, Homo sapiens
MLGLVMSCPDRTLVTKVRSHPSGNQHRPTRGGSRSFHCRRGVRPRPAALRVLPRCPAFSDAACPAAMRPPQCLLHTPSLASPLLLLLLWLLGGGVG

SEQ ID NO: 3 - protein, 606 aa, Artificial sequence
  Acetylcholinesterase fused to the C-terminal ER-retention signal (hAChE-R ER) polypeptide sequence
MRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATGMQGPAGSGWEEGSGSPPGVTPLFSPSEKDEL

SEQ ID NO: 4 - protein, 26 aa, Artificial sequence
  hARP - an active portion of AChE-R
GMQGPAGSGWEEGSGSPPGVTPLFSP

SEQ ID NO: 5 - protein, 30 aa, Artificial sequence
  mARP - an active portion of AChE-R
GRRMEWGEQGMHKAARVGRRGERWGAKHRV

(19) United States
(12) Patent Application Publication
(10) Pub. No.: US 2013/0164288 A1
(43) Pub. Date: Jun. 27, 2013

(54) READTHROUGH ACETYLCHOLINESTERASE (ACHE-R) FOR TREATING OR PREVENTING PARKINSON'S DISEASE

(71) Applicants: Hermona Soreq, Jerusalem (IL); Ilya Ruderfer, Carmiel (IL); Yoseph Shaaltiel, Kibbutz HaSolelim (IL)

(72) Inventors: Hermona Soreq, Jerusalem (IL); Ilya Ruderfer, Carmiel (IL); Yoseph Shaaltiel, Kibbutz HaSolelim (IL)

(73) Assignees: PROTALIX LTD., Carmiel (IL); YISSUM RESEARCH DEVELOPMENT COMPANY OF THE HEBREW UNIVERSITY OF JERUSALEM LTD., Jerusalem (IL)

(21) Appl. No.: 13/820,525

(22) PCT Filed: Sep. 7, 2011
(86) PCT No.: PCT/IL2011/000721

§ 371 Date: Mar. 4, 2013

Related U.S. Application Data
(60) Provisional application No. 61/380,353, filed on Sep. 7, 2010.

(51) Int. Cl. C12N 9/18; C12N 9/96

(52) U.S. Cl. CPC C12N 9/18; C12N 9/96
USPC 424/134.1; 424/94.6; 424/94.3

(57) ABSTRACT

A method of treating or preventing Parkinson's disease in a subject in need thereof is disclosed. The method comprises administering to the subject a therapeutically effective amount of AChE-R, wherein the AChE-R is devoid of an N-terminal extension. An additional method of treating or preventing Parkinson's disease in a subject is disclosed. The method comprises administering to the subject a therapeutically effective amount of AChE-R, wherein the AChE-R comprises a modification for increasing bioavailability.

### FIELD AND BACKGROUND OF THE INVENTION

The present invention is of materials and methods for treating or preventing Parkinson's disease, specifically, the present invention relates to the use of AChE-R for treating or preventing Parkinson's disease.

Parkinson's disease (PD) is an age-related disorder characterized by progressive loss of dopamine producing neurons in the substantia nigra of the midbrain, which in turn leads to progressive loss of motor functions manifested through symptoms such as to tremor, rigidity and ataxia. Parkinson's disease can be treated by administration of pharmacological doses of the precursor of dopamine, L-DOPA (Marsden, Trends Neurosci. 9:512, 1986; Vinken et al., in Handbook of Clinical Neurology p. 185, Elsevier, Amsterdam, 1986). Although such treatment is effective in early stage Parkinson's patients, progressive loss of substantia nigra cells eventually leads to an inability of remaining cells to synthesize sufficient dopamine from the administered precursor and to diminishing pharmacogenic effect.

Alternative splicing is a brain-prevalent process for expanding the transcriptome repertoire by generating from one gene, multiple mRNAs that encode functionally different proteins with distinct tissue specificities and sometimes antagonistic functions. Disrupted alternative splicing associates with several neurodegenerative disorders (e.g. Presenilin 2 splice variant in Alzheimer's disease, exon 10 inclusion in tau mRNA of frontotemporal dementia with Parkinsonism or splice variants of Parkin and ania6 in PD).

PD is associated with impairments in the dopaminergic-cholinergic balance and with environmental causes, among them exposure to acetylcholinesterase inhibitors (anti-AChEs) such as organophosphate (OP) pesticides^{9,10}. Of note, exposure to anti-AChEs causes a splice shift from the synaptic form of AChE, AChE-S to the monomeric readthrough variant AChE-R^{11,12}. Inherited impairments in the splice shift to AChE-R increase the risk of PD under exposure to anti-AChEs¹³. Transgenic overexpression of AChE-R confers resistance, and of AChE-S-sucsceptibility, to the dopaminergic neurotoxin 1-methyl-4-phenyl-1,2,3,6-tetrahydropyridine (MPTP) with or without co-exposure to organophosphate anti-cholinesterases¹⁴. MPTP damages midbrain dopaminergic neurons projecting to the Parkinsonian caudate-putamen (CPu) and the pre-frontal cortex (PFC)¹⁵. However, until presently it remained unclear if the AChE-R inducible protection originated in the brain or in peripheral tissues, whether these effects are limited to AChE transcripts and what are the underlying molecular mechanisms.

Additional background art includes WO2007/049281 which teaches AChE-R comprising an N terminal extension for the treatment of neurodegenerative diseases.

### SUMMARY OF THE INVENTION

According to one aspect there is provided a method of treating or preventing Parkinson's disease in a subject in need thereof, the method comprising administering to the subject a therapeutically effective amount of AChE-R, wherein the AChE-R is devoid of an N-terminal extension to thereby treat the Parkinson's disease in the subject.

According to an additional aspect there is provided a method of treating or preventing Parkinson's disease in a subject in need thereof, the method comprising administering to the subject a therapeutically effective amount of AChE-R, wherein the AChE-R comprises a modification for increasing bioavailability, thereby treating the Parkinson's disease in the subject.

According to one embodiment, the AChE-R comprise recombinant AChE-R.

According to one embodiment, the recombinant AChE-R is plant produced AChE-R.

According to one embodiment, the AChE-R is as set forth in SEQ ID NOs. 1 and 3.

According to one embodiment, the administering is peripherally administering.

According to one embodiment, the AChE-R is devoid of an N-terminal extension.

According to one embodiment, the AChE-R comprises an N-terminal extension.

According to one embodiment, the N-terminal extension is at least 90% homologous to SEQ ID NO: 2.

According to one embodiment, the AChE-R comprises recombinant AChE-R.

According to one embodiment, the recombinant AChE-R is plant produced AChE-R.

According to one embodiment, the modification comprises attachment to a heterologous polypeptide.

According to one embodiment, the heterologous polypeptide is selected from the group consisting of human serum albumin, immunoglobulin, and transferrin.

According to one embodiment, the immunoglobulin comprises an Fc domain.

According to one embodiment, the modification comprises attachment to a polymer.

According to one embodiment, the polymer is selected from the group consisting of a polycationic polymer, a non-ionic water-soluble polymer, a polyether polymer and a biocompatible polymer.

According to one embodiment, the polymer is poly(ethylene glycol).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C illustrate modified expression of splicing-related transcripts following MPTP exposure. (A) Log 2-fold changes of all nuclear mRNA splicing probe sets between MPTP-exposed and naive FVB/N mice. Colors indicate fold changes and numerals provide entries into the corresponding UniGene (Wheeler et al., 2005) clusters (Table 2). Circles represent significant changes according to the Affymetrix change algorithm. (B) Signal correlations among arrays for probes shown in A, for two biological replicates of naive (F1,F2) and two replicates of MPTP-exposed mice (FM1,FM2). (C) Hierarchical parent-child order of individual GO terms. Boxes show cumulative distribution function (CDF) of log 2 fold-changes for associated probes (green) vs. all transcripts on the array (black). Arrows represent direction of change. C: Continuous method, D: Discrete method; red vertical lines represent individual transcripts exceeding the 2-fold threshold. Numbers within parentheses are GO IDs.

FIGS. 2A-G illustrate that TgS, more than TgR, brain regions show larger gene expression differences compared to wild type mice. Inset: The studied brain regions (A) Volcano plots of 2-way ANOVA tests (Inc) comparing CPu and PFC gene expression in TgS and TgR to naïve FVB/N. (B) Volcano profiles before and after MPTP exposure, within TgR, TgS and FVB/N in the CPu and in the PFC. Axes: ANOVA P-values as a function of fold changes. Dashed lines: p<0.05 (y axis) and Fold change>|1.5| (x axis). (C-D) qRT-PCR validation of CPu microarrays. Y axis: fold change. Asterisks: P<0.05. (E) Experimental scheme. CPu and PFC of FVB/N, TgS and TgR mice were isolated from naïve and MPTP treated-mice for gene expression analyses. (F-G) qRT-PCR validation of CPu microarrays. Y axis: fold change. Asterisks: P<0.05.

FIGS. 3A-E illustrate MPTP exposure induces concerted changes in spliceosomal transcripts. (A) 3D depiction of expression patterns of two triplets of transcripts. The axes share the same scale and represent signal levels of each GeneChip transcript. (B) Correlation matrix of mRNA-splicing related transcripts between all arrays (3 strains×2 treatments×2 biological replicates). (C) Average correlations over biological duplicates. F, R, S: naive FVB/N, TgR and TgS mice; the M prefix denotes MPTP. (D) Euclidian distances in “probe space” between spliceosomal configurations for each strain and between naive and MPTP-exposed animals. Vertex distances are proportional to the distance in the 140-dimensional “probe space”. (E) Clustering factors for hierarchically related GO biological process terms, starting from the entire set of transcripts and “zooming-in” on splice site selection probes.

FIGS. 4A-F illustrate prophylaxis and therapeutic protection by rhAChE-R from lethal DEPQ challenge. (A) Pharmacokinetics of rhAChE-R. Plasma AChE activity is presented as μmole substrate hydrolyzed/min/ml±stdev (n=3-13 mice for each time point) following i.v. injection with 1 nmole/mouse of rhAChE-R(N=9). Mice were sacrificed the noted time in hours following administration. (B) AChE activity in the gastrocnemius muscle (C) parietal cortex and (D) hippocampus (nmole substrate hydrolyzed/min/mg±stdev), compared to saline-injected control mice. (E) Mice were injected i.v. (Mandel et al., 2000; Miller et al., 2004) with 3 different doses of rhAChE-R followed after 2 min by i.v. injection of DEPQ (40 μg/Kg) to yield the indicated enzyme/OP ratios. Circles represent individual mice. See text for symptoms scoring. Star: the relevant enzyme/OP ratio (0.26) that was used at the s.c experiment. (F) Mice to were injected s.c. with DEPQ (75 μg/Kg) followed after 10, 15 or 20 min by i.v. injection of rhAChE-R (50 nmole/Kg) (n=8 mice at each time point).

FIGS. 5A-C illustrate enforced expression of AChE variants modifies the brain's gene expression and MPTP response. Top Scheme (A) The synaptic AChE-S splice variant (in red) and the monomeric soluble AChE-R variant (in blue). Exon 6 and pseuodointron 4 encode the variant-specific —S and —R C-termini, respectively. (B) Horizontal brain section with Niss1 staining of the PFC and the CPu. (C) Probe sets changed significantly between strains in the CPu and PFC. Top: Numbers of probe sets changed significantly (p<0.05, fold change>1.5) in the TgS and TgR naïve mice as compared to naive FVB/N mice. Green columns: changes in the CPu as compared to the PFC in each strain. Asterisks: p<0.05. Bottom: Numbers of probe sets changed significantly (p<0.05, fold change>1.5) in the TgS, TgR and FVB/N mice following MPTP exposure.

FIGS. 6A-C illustrate MPTP-induced changes in ASF/SF2 RNA and protein. (A)

Transcript composition depicting location of Affymetrix probe #1452430 (circle with #44 in 3 A), upper left: scanned GeneChip images, and signal magnitudes defined as perfect or mismatch (PM-MM values) for each probe pair in the probe set (www.affymetrix.com.). Lower left: RT-PCR products of primers spanning exons 1-2. C: Control, S: Saline, M: MPTP. (B) Confocal images of fluorescent ASF/SF2 immunolabeling of representative saline-treated and MPTP-exposed cortices from FVB/N mice at 63× without (top), and with (bottom) a digital 3.3× zoom. Data in A and B are for FVB/N mice. (C) Confocal images of fluorescent ASF/SF2 immunolabeling from representative saline-treated and MPTP-exposed cortices of transgenic strains. Insets show RT-PCR products for ASF/SF2. In each inset, the two leftmost bands are for two saline-injected prefrontal cortex (PFC) pools, and the two rightmost bands are for MPTP-injected PFC pools.

### DESCRIPTION OF SPECIFIC EMBODIMENTS OF THE INVENTION

The present invention is of materials and methods for treating or preventing Parkinson's disease, specifically, the present invention relates to the use of AChE-R for treating or preventing Parkinson's disease.

The principles and operation of the invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

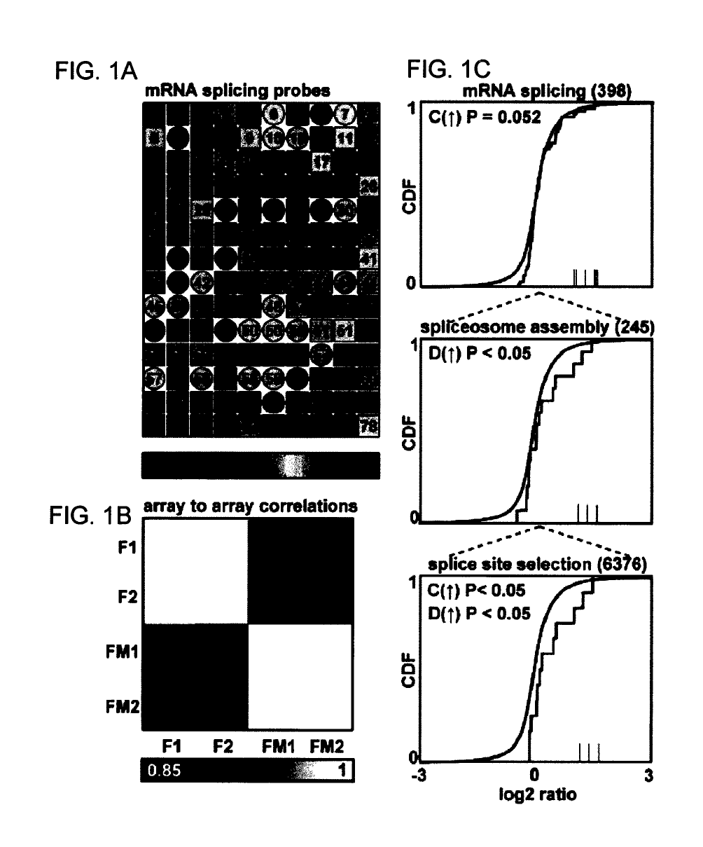

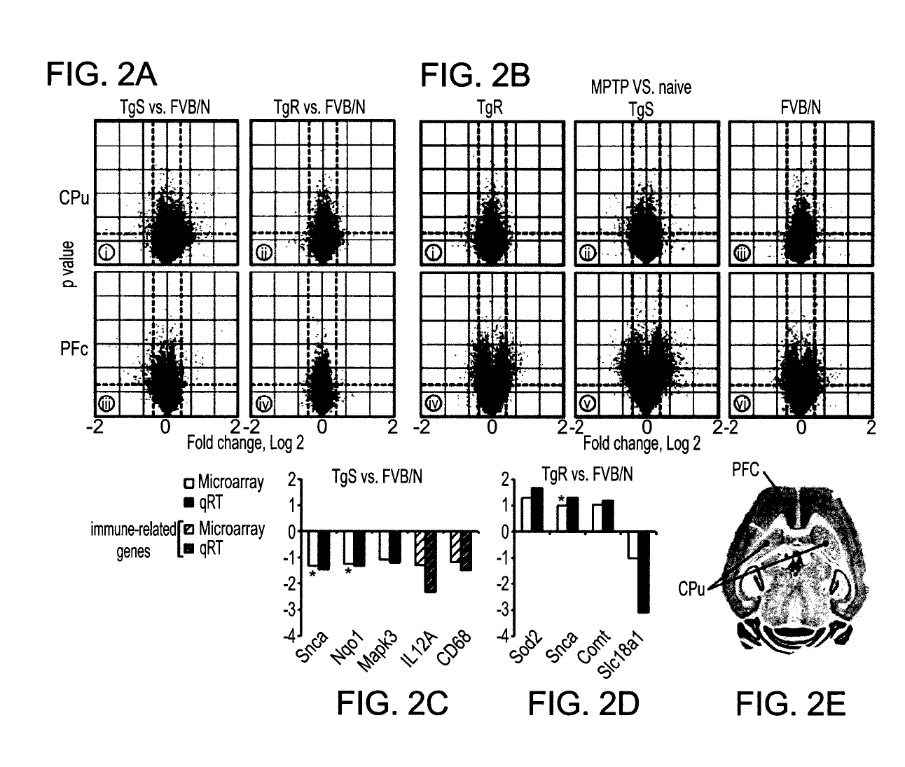

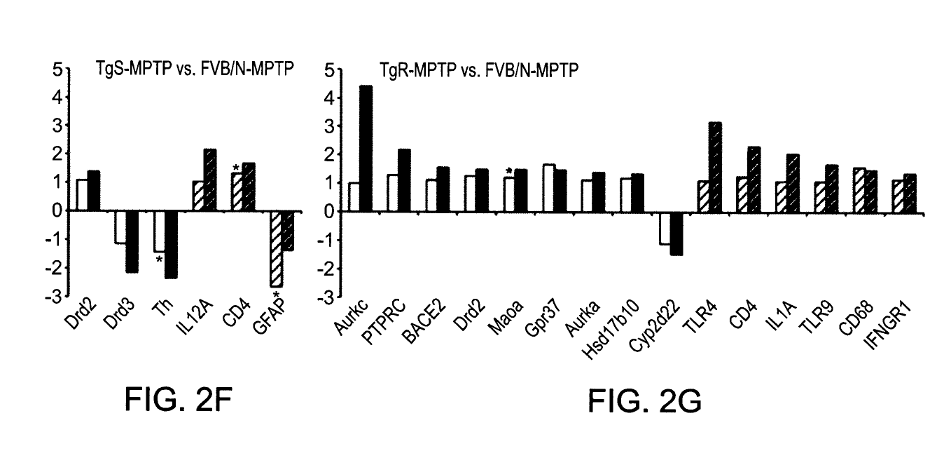

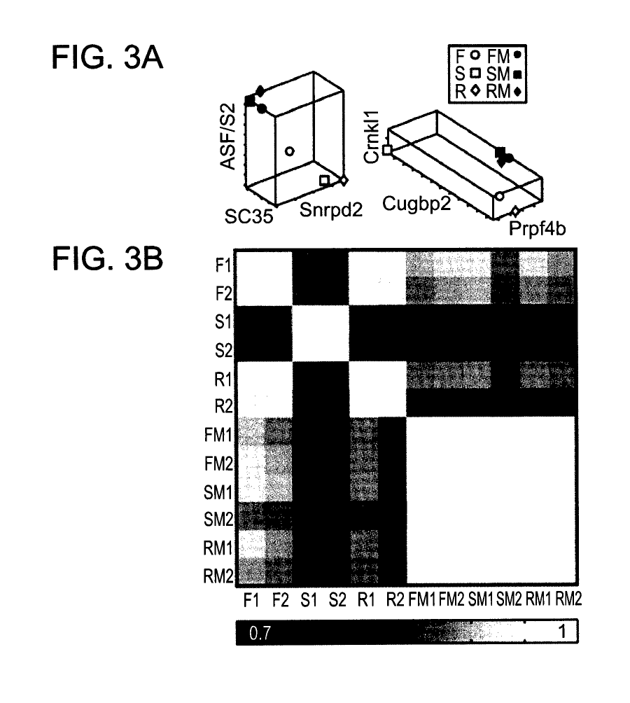

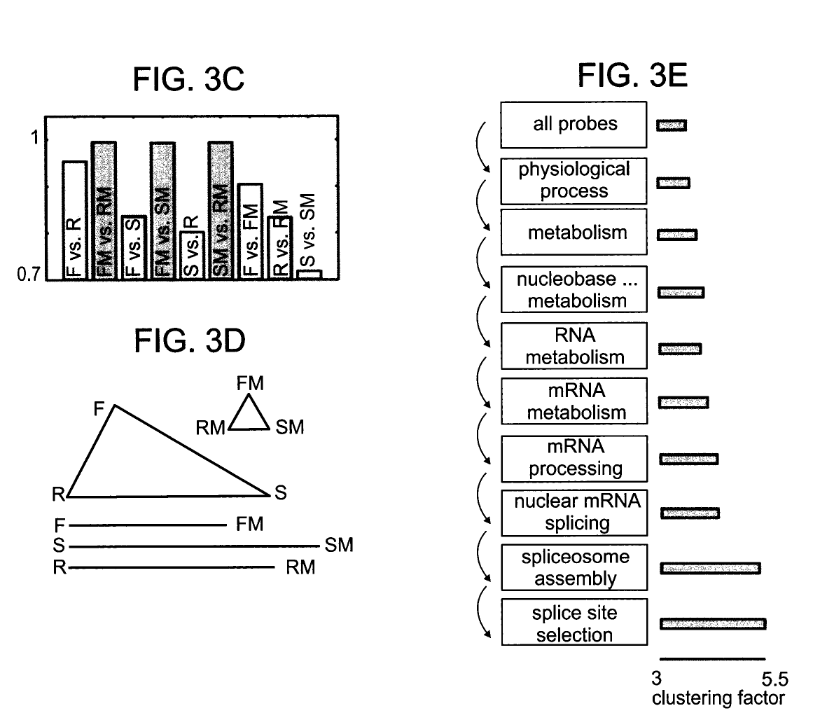

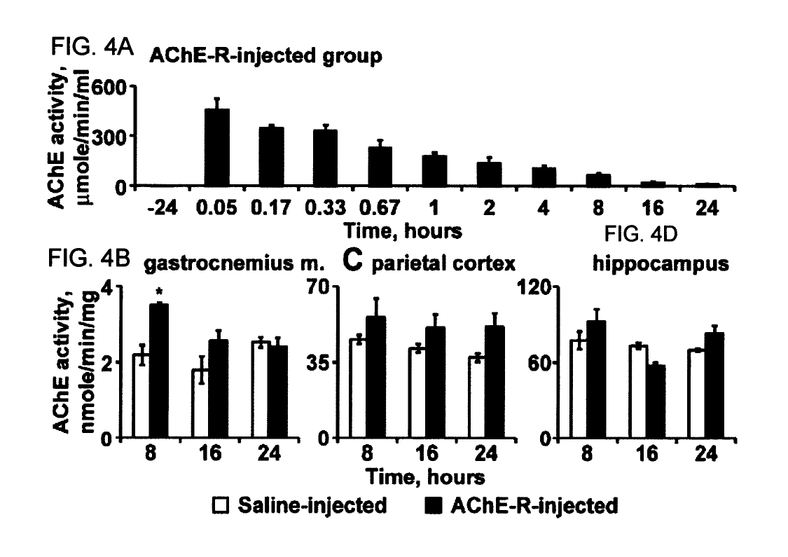

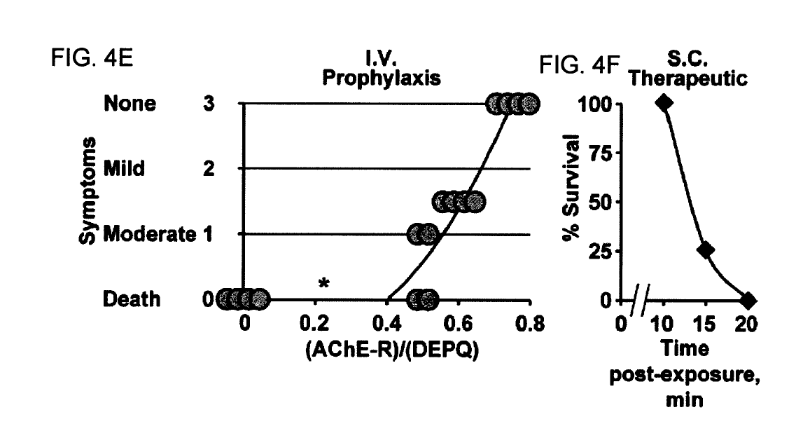

It has previously been shown that inherited AChE-R excess protects from, whereas AChE-S exacerbates Parkinsonism in mice exposed to the dopaminergic neurotoxin 1-methyl-4-phenyl-1,2,3,6-tetrahydropyridine (MPTP) or to poisonous AChE inhibitors [Ben Shaul et al, Eur J Neurosci 2006; 23: 2915-22]. However until presently, it was not known whether peripheral treatment with AChE-R would be beneficial for the treatment of Parkinson's disease. The present inventors have now found from analysis of prefrontal cortex (PFC) expression patterns that MPTP triggers reproducible prominent changes of multiple splicing-related categories in this brain area of wild-type and transgenic AChE over-expressors (FIGS. 1A-C). Specifically, mice with enforced AChE-R over-expression showed fewer total changes yet abundant alternative splicing events compared to AChE-S over-expressors (FIGS. 2A-G). Indicating functional relevance of these splicing changes, the present inventors found larger impairments of ASF/SF2 (an exemplary splice related transcript) expression as well as nuclear clustering in the PFC of AChE-S over-expressors (FIGS. 3A-C and 6A-C). To test whether peripheral and/or brain-limited processes were involved, the present inventors intravenously injected mice with plant-produced, highly purified recombinant human AChE-R (FIGS. 4A-D). This peripheral treatment remarkably induced multiple gene expression changes in the Parkinsonian Caudate-Putamen (CPu) brain region.

All these findings support the use of AChE-R for the treatment and prevention of Parkinson's disease.

Thus, according to an aspect of the invention, there is provided a method of treating or preventing Parkinson's disease in a subject in need thereof, the method comprising administering to the subject a therapeutically effective amount of AChE-R, to thereby treat the Parkinson's disease in the subject.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein the phrase “a subject in need thereof” refers to a human or animal subject.

As used herein the term “AChE-R” refers to the acetylcholinesterase readthrough variant such as set forth in GenBank Accession Number DQ140347 (e.g., SEQ ID NOs: 1 or 3) or an active portion thereof e.g., ARP set forth in SEQ ID NOs. 4 and 5.

The AChE-R of the present invention may comprise or be devoid of an N-terminal extension. Such an N-terminal extension is preferably at least 70% homologous to SEQ ID NO: 2, or at least 80% homologous to SEQ ID NO: 2, or more preferably at least 90% homologous to SEQ ID NO: 2, or more preferably at least 95% homologous and even more preferably is as set forth in SEQ ID NO: 2.

The AChE-R of the present invention may be naturally expressed (i.e., purified), synthetic or recombinantly produced such as in bacteria, yeast, cell-lines, transgenic animal (e.g., see U.S. Pat. No. 5,932,780, herein incorporated by reference in its entirety).

Recombinant techniques are preferably used to generate the AChE-R since these techniques are better suited for generation of relatively long polypeptides (e.g., longer than 20 amino acids) and large amounts thereof. Such recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

To produce AChE-R using recombinant technology, polynucleotide encoding a polypeptide of the present invention is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

As used herein, the phrase “cis acting regulatory element” refers to a polynucleotide sequence, preferably a promoter, which binds a trans acting regulator and regulates the transcription of a coding sequence located downstream thereto.

As used herein, the phrase “operably linked” refers to a functional positioning of the cis-regulatory element (e.g., promoter) so as to allow regulating expression of the selected nucleic acid sequence. For example, a promoter sequence may be located upstream of the selected nucleic acid sequence in terms of the direction of transcription and translation.

The expression vector of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

A variety of prokaryotic or eukaryotic cells can be used as host-expression systems to express the polypeptides of the present invention. These include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

Polyadenylation sequences can also be added to the expression vector in order to increase the translation efficiency of a polypeptide expressed from the expression vector of the present invention. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic to replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

According to a preferred embodiment, plant cells are used to express the polypeptides of the present invention.

As used herein the term “plant” refers to a whole plant, portions thereof, plant cell, plant cell culture or plant cell suspension. The transformed or transfected plant of the present invention may be any monocotyledonous or dicotyledonous plant or plant cell, as well as, coniferous plants, moss, algae, monocot or dicot and other plants listed in wwwdotnationmasterdotcom/encyclopedia/Plantae. Examples of monocotyledonous plants include, which can be used in accordance with the present invention include, but are not limited to, corn, cereals, grains, grasses, and rice. Examples of dicotyledonous plants which can be used in accordance with the present invention include, but are not limited to, tobacco, tomatoes, carrots, potatoes, and legumes including soybean and alfalfa.

According to this embodiment of this aspect of the present invention, the nucleic acid sequence encoding the AChE-R polypeptides of the present invention may be altered, to further improve expression levels in plant expression system. AChE-R may be modified in accordance with the preferred codon usage for plant expression. Increased expression of the AChE-R polypeptides in plants may be obtained by utilizing a modified or derivative nucleotide sequence. Examples of such sequence modifications include, but are not limited to, an altered G/C content to more closely approach that typically found in plants, and the removal of codons atypically found in plants commonly referred to as codon optimization.

The phrase “codon optimization” refers to the selection of appropriate DNA nucleotides for use within a structural gene or fragment thereof that approaches codon usage within a plant.

Preferably, the promoter in the nucleic acid construct of the present invention is a plant promoter which serves for directing expression of the nucleic acid molecule within plant cells.

As used herein the phrase “plant promoter” refers to a promoter which can direct transcription of the polynucleotide sequence in plant cells. Such a promoter can be derived from a plant, bacterial, viral, fungal or animal origin. The promoter may be constitutive, i.e., capable of directing high level of gene expression in a plurality of plant tissues, tissue specific, i.e., capable of directing gene expression in a particular plant tissue or tissues, developmentally regulated, inducible, i.e., capable of directing gene expression under a stimulus, or chimeric.

Examples of constitutive plant promoters include, but are not limited to CaMV35S and CaMV19S promoters, FMV34S promoter, sugarcane bacilliform badnavirus promoter, CsVMV promoter, Arabidpsis ACT2/ACT8 actin promoter, Arabidpsis ubiquitin UBQ 1 promoter, barley leaf thionin BTH6 promoter, and rice actin promoter.

In the case where a tissue-specific promoter is used, protein expression is particularly high in the tissue from which extraction of the protein is desired. Depending on the desired tissue, expression may be targeted to the endosperm, aleurone layer, embryo (or its parts as scutellum and cotyledons), pericarp, stem, leaves, tubers, trichomes, seeds, roots, etc. Examples of tissue specific promoters include, but are not limited to bean phaseolin storage protein promoter, DLEC promoter, PHS β promoter, zein storage protein promoter, conglutin gamma promoter from soybean, AT2S1 gene promoter, ACT11 actin promoter from Arabidpsis, napA promoter from Brassica napus and potato patatin gene promoter.

An inducible promoter is a promoter induced by a specific stimulus such as stress conditions comprising, for example, light, temperature, chemicals, drought, high salinity, osmotic shock, oxidant conditions or in case of pathogenicity. Usually the promoter is induced before the plant is harvested and as such is referred to as a pre-harvest promoter. Examples of inducible pre-harvest promoters include, but are not limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr2O3J and str246C active in pathogenic stress.

The inducible promoter may also be an inducible post-harvest promoter e.g. the inducible MeGA.™ promoter (U.S. Pat. No. 5,689,056). The preferred signal utilized for the rapid induction of the MeGA™ promoter is a localized wound after the plant has been harvested.

As mentioned herein above, the nucleic acid construct of the present invention may also comprise an additional nucleic acid sequence encoding a signal peptide that allows transport of the AChE-R polypeptides in-frame fused thereto to a sub-cellular organelle within the plant, as desired. Examples of subcellular organelles of plant cells include, but are not limited to, leucoplasts, chloroplasts, chromoplasts, mitochondria, nuclei, peroxisomes, endoplasmic reticulum and vacuoles.

Compartmentalization of the AChE-R recombinant protein within the plant cell followed by its secretion is one pre-requisite of making the product easily purifiable. It was shown that targeting a recombinant protein to the endoplasmic reticulum by fusion with an appropriate signal peptide allows the fused polypeptide to be targeted to a secretory pathway. Accumulation of the protein in a subcellular organelle of the cell may also be preferred to allow the protein to be stored in relatively high concentrations without being exposed to degrading compounds present in the vacuole, for example. Signaling sequences may be derived from plants such as wheat, barley, cotton, rice, soy, and potato.

Exemplary signal peptides that may be used herein include the tobacco pathogenesis related protein (PR-S) signal sequence (Sijmons et al., 1990, Bio/technology, 8:217-221), lectin signal sequence (Boehn et al., 2000, Transgenic Res, 9(6):477-86), signal sequence from the hydroxyproline-rich glycoprotein from Phaseolus vulgaris (Yan et al., 1997, Plant Phyiol. 115(3):915-24 and Corbin et al., 1987, Mol Cell Biol 7(12):4337-44), potato patatin signal sequence (Iturriaga, G et al., 1989, Plant Cell 1:381-390 and Bevan et al., 1986, Nuc. Acids Res. 41:4625-4638.) and the barley alpha amylase signal sequence (Rasmussen and Johansson, 1992, Plant Mol. Biol. 18(2):423-7). Such targeting signals may be cleaved in vivo from the AChE variat sequence, which is typically the case when an apoplast targeting signal, such as the tobacco pathogenesis related protein-S(PR-S) signal sequence (Sijmons et al., 1990, Bio/technology, 8:217-221) is used.

Other signal sequences which may also be used in accordance with this aspect of the present invention include signal retention sequences. Use of these sequences result in increased accumulation in a particular location and therefore may provide for easier purification of the AChE polypeptides of the present invention.

For example, Pat. Appl. No. 20050039235 teaches the use of signal and retention polypeptides for targeting recombinant insulin to the ER or in an ER derived storage vesicle (e.g. an oil body) in plant cells thereby increasing the accumulation of insulin in seeds.

Examples of ER retention motifs include KDEL, HDEL, DDEL, ADEL and SDEL sequences.

Yet another important strategy to facilitate purification is to fuse the recombinant AChE-R of the present invention with an affinity tag by including a sequence of the tag in the nucleic acid construct of the present invention. This method is widely utilized for in vitro purification of proteins. Exemplary purification tags for purposes of the invention include but are not limited to polyhistidine, V5, myc, protein A, gluthatione-5-fransferase, maltose binding protein (MBP) and cellulose-binding domain (CBD) [Sassenfeld, 1990, TIBTECH, 8, 88-9]. In the case of CBD fusion proteins, the AChE polypeptides are fused to a substrate-binding region of a polysaccharidase (cellulases, chitinases and amylases, as well as xylanases and the beta.-1,4 glycanases). The affinity matrix containing the substrate such as cellulose can be employed to immobilize the AChE-R polypeptides. The AChE-R polypeptides can be removed from the matrix using a protease cleavage site.

The nucleic acid construct of the present invention may also comprise a sequence that aids in proteolytic cleavage, e.g., a thrombin cleavage sequence. Such a sequence may permit the AChE polypeptides to be separated from an attached co-translated sequence such as the ER retention sequences described above.

The nucleic acid construct of the present invention may be capable of integrating into the plant genome and as such would direct the expression of a AChE-R polypeptide coding sequence. Alternatively, the nucleic acid construct may be an episomal construct directing a transient AChE-R coding sequence expression.

The above-described nucleic acid construct can be used for producing AChE-R in plants. This can be performed by (a) introducing the nucleic acid construct described hereinabove into a plant; (b) cultivating the plant under conditions which allow expression of the acetylcholinesterase; and (c) recovering the acetylcholinesterase from the plant.

There are various methods of introducing foreign genes into plants (Potrykus, I., Annu. Rev. Plant. Physiol., Plant. Mol. Biol. (1991) 42:205-225; Shimamoto et al., Nature (1989) 338:274-276).

The principle methods of effecting stable integration of exogenous nucleic acid sequence into plant genomic DNA include two main approaches:

(i) Agrobacterium-mediated gene transfer: [Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S, and Arntzen, C. J., Butterworth Publishers, Boston, Mass. (1989) p. 93-112].

(ii) direct DNA uptake: [Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074 DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719].

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in [Plant Molecular Biology Manual A5, Kluwer Academic Publishers, Dordrecht (1988) p. 1-9. A supplementary approach] employs the Agrobacterium delivery system in combination with vacuum infiltration. The Agrobacterium system is especially viable in the creation of transgenic dicotyledenous plants (as described in the Examples section which follows).

Additional methods of transgenic plant propagation and transformation are described in U.S. Pat. Nos. 6,610,909 to Oglevee-O'Donavan et al, and 6,384,301 to Martinell et al, both incorporated herein by reference.

There are various methods of direct DNA transfer into plant cells. In electroporation, the protoplasts are briefly exposed to a strong electric field. In microinjection, the DNA (i.e. nucleic acid construct encoding the AChE variants of the present invention) is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

The AChE-R may be clinically used following recovery. The term “recovery” refers to at least a partial purification to yield a plant extract, homogenate, fraction of plant homogenate or the like. Partial purification may comprise, but is not limited to disrupting plant cellular structures thereby creating a composition comprising soluble plant components, and insoluble plant components which may be separated for example, but not limited to, by centrifugation, filtration or a combination thereof. In this regard, proteins secreted within the extracellular space of leaf or other tissues could be readily obtained using vacuum or centrifugal extraction, or tissues could be extracted under pressure by passage through rollers or grinding or the like to squeeze or liberate to the protein free from within the extracellular space. Minimal recovery could also involve preparation of crude extracts of AChE-R variants, since these preparations would have negligible contamination from secondary plant products. Further, minimal recovery may involve methods such as those employed for the preparation of F1P as disclosed in Woodleif et al., Tobacco Sci. 25, 83-86 (1981). These methods include aqueous extraction of soluble protein from green tobacco leaves by precipitation with any suitable salt, for example but not limited to KHSO₄. Other methods may include large scale maceration and juice extraction in order to permit the direct use of the extract.

Alternatively, recovery of the AChE-R polypeptides from the plant (whole plant) or plant culture can be effected using more sophisticated purification methods which are well known in the art. For example, collection and/or purification of AChE-R from plant cells or plants can depend upon the particular expression system and the expressed sequence. Separation and purification techniques can include, for example, ultra filtration, affinity chromatography and or electrophoresis. In particular instances, molecular biological techniques known to those skilled in the art can be utilized to produce variants having one or more heterologous peptides which can assist in protein purification (purification tags, as described above). Such heterologous peptides can be retained in the final functional protein or can be removed during or subsequent to the collection/isolation/purification processing.

For clinical use, the AChE-R is preferably highly purified such as to medical grade purity (above 95%, more preferably 99% or more).

Recombinant proteins of the present invention may be modified prior to or following recovery as further described hereinbelow.

It will be appreciated that production of active AChE-R requires post translational modifications, i.e. glycosylation. AChE-R is a glycoprotein comprising 3 potential N-glycosylation sites. Glycosylation at at least 2 of the sites is important for effective biosynthesis and secretion [Yelan et al, Biochem J. 1993 December 15; 296(Pt 3): 649-656]. Although plants glycosylate human proteins at the correct position, the composition of fully processed complex plant glycans differ from mammalian N-linked glycans. Plant glycans do not have the terminal sialic acid residue or galactose residues to common in animal glycans and often contain a xylose or fucose residue with a linkage that is generally not found in mammals (Jenkins et al., 14 Nature Biotech 975-981 (1996); Chrispeels and Faye in transgenic plants pp. 99-114 (Owen, M. and Pen, J. eds. Wiley & Sons, N.Y. 1996; Russell 240 Cum Top. Microbio. Immunol. (1999). Specifically, plants comprise additional beta 1-2 linked xylosyl- and alpha 1-3 linked fucosyl-residues which are not found in mammals. Conversely they do not comprise fucosyl-1-6-residues which are present in mammals.

The presence of xylose/fucose residues has been associated with antigenic responses (Chrispeels and Faye, supra). Galactose residues are thought to play a role in IgG-complement interactions. Also, sialic acid residues are required for pharmacokinetic reasons extending the in-vivo half-life of the associated polypeptide in the human recipient. Thus, the present invention contemplates the use of various strategies to address the issue of “humanization” of glycans of AChE-R synthesized in plants. Such strategies are known in the art—see e.g. U.S. Pat. Appl. 20030033637.

AChE-R of some embodiments of the invention may be chemically modified for increasing bioavailability.

Thus for example, the present invention contemplates modifications wherein the AChE-R polypeptide is linked to a polymer. The polymer selected is usually modified to have a single reactive group, such as an active ester for acylation or an aldehyde for alkylation, so that the degree of modification may be controlled. Included within the scope of polymers is a mixture of polymers. Preferably, for therapeutic use of the end-product preparation, the polymer will be pharmaceutically acceptable.

The polymer or mixture thereof may be selected from the group consisting of, for example, polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In further still embodiments, the AChE-R polypeptide is modified by PEGylation, HESylation CTP (C terminal peptide), crosslinking to albumin, encapsulation, modification with polysaccharide or polysaccharide alteration. The modification can be to any amino acid residue in the AChE-R polypeptide.

According to one embodiment the modification is to the N or C-terminal amino acid of the AChE-R polypeptide. This may be effected either directly or by way of coupling to the thiol group of a cysteine residue added to the N or C-terminus or a linker added to the N or C-terminus such as Ttds. In further embodiments, the N or C-terminus of the AChE-R polypeptide comprises a cysteine residue to which a protecting group is coupled to the N-terminal amino group of the cysteine residue and the cysteine thiolate group is derivatized with a functional group such as N-ethylmaleimide, PEG group, HESylated CTP.

It is well known that the properties of certain proteins can be modulated by attachment of polyethylene glycol (PEG) polymers, which increases the hydrodynamic volume of the protein and thereby slows its clearance by kidney filtration. (See, for example, Clark et al., J. Biol. Chem. 271: 21969-21977 (1996). Therefore, it is envisioned that the core peptide residues can be PEGylated to provide enhanced therapeutic benefits such as, for example, increased efficacy by extending half-life in vivo. Thus, PEGylating the AChE-R polypeptide will improve the pharmacokinetics and pharmacodynamics of the AChE-R polypeptide.

PEGylation methods are well known in the literature and described in the following references, each of which is incorporated herein by reference: Lu et al., Int. J. Pept. Protein Res. 43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., Int. J. Hematol. 68: 1-18 (1998); Roberts et al., J. Pharm. Sci. 87: 1440-45 (1998); and Tan et al., Protein Expr. Purif. 12: 45-52 (1998). Polyethylene glycol or PEG is meant to encompass any of the forms of PEG that have been used to derivatize other proteins, including, but not limited to, mono-(C.sub.1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG.sub.2-NHS-40k (Nektar); mPEG₂-MAL-40k (Nektar), SUNBRIGHT GL2-400MA ((PEG).sub.240 to kDa) (NOF Corporation, Tokyo), SUNBRIGHT ME-200MA (PEG20 kDa) (NOF Corporation, Tokyo). The PEG groups are generally attached to the AChE-R polypeptide via acylation, amidation, thioetherification or reductive alkylation through a reactive group on the PEG moiety (for example, an aldehyde, amino, carboxyl or thiol group) to a reactive group on the AChE-R polypeptide (for example, an aldehyde, amino, carboxyl or thiol group).

The PEG molecule(s) may be covalently attached to any Lys or Cys residue at any position in the AChE-R polypeptide. Other amino acids that can be used are Tyr and His. Optional are also amino acids with a Carboxylic side chain. The AChE-R polypeptide described herein can be PEGylated directly to any amino acid at the N-terminus by way of the N-terminal amino group. A “linker arm” may be added to the AChE-R polypeptide to facilitate PEGylation. PEGylation at the thiol side-chain of cysteine has been widely reported (See, e.g., Caliceti & Veronese, Adv. Drug Deliv. Rev. 55: 1261-77 (2003)). If there is no cysteine residue in the AChE-R polypeptide, a cysteine residue can be introduced through substitution or by adding a cysteine to the N-terminal amino acid. Other options include reagents that add thiols to polypeptides, such as Traut's reagents and SATA.

In particular aspects, the PEG molecule is branched while in other aspects, the PEG molecule may be linear. In particular aspects, the PEG molecule is between 1 kDa and 150 kDa in molecular weight. More particularly, the PEG molecule is between 1 kDa and 100 kDa in molecular weight. In further aspects, the PEG molecule is selected from 5, 10, 20, 30, 40, 50 and 60 kDa.

A useful strategy for the PEGylation of AChE-R polypeptide consists of combining, through forming a conjugate linkage in solution, a peptide, and a PEG moiety, each bearing a special functionality that is mutually reactive toward the other. The AChE-R polypeptide can be easily prepared by recombinant means as described above.

According to one embodiment, the PEG is “preactivated” prior to attachment to the AChE-R polypeptide. For example, carboxyl terminated PEGs may be transformed to NHS esters for activation making them more reactive towards lysines and N-terminals.

According to another embodiment, the AChE-R polypeptide is “preactivated” with an appropriate functional group at a specific site. Conjugation of the AChE-R polypeptide with PEG may take place in aqueous phase or organic co-solvents and can be easily monitored by SDS-PAGE, isoelectric focusing (IEF), SEC and mass spectrometry. The PEGylated AChE-R polypeptide is then purified. Small PEGs may be removed by ultra-filtration. Larger PEGs are typically purified using anion chromatography, cation chromatography or affinity chromatography. Characterization of the PEGylated polypeptide may be carried out by analytical HPLC, amino acid analysis, IEF, analysis of enzymatic activity, electrophoresis, analysis of PEG:protein ratio, laser desorption mass spectrometry and electrospray mass spectrometry.

An exemplary method for attachment of PEG chains to primary amines in AChE-R may be performed using the PEGylation reagent a-Methoxy-PEG10K-w-NHS esters (Rapp-Polymere, 12-10000-35). AChE-R (−1 mg/ml) is incubated with the PEGylation reagent at a ratio of about 1:32 (w/w) [ACNE]/[PEG], shaking for 1 hour at room temperature and overnight at 2-8° C.

Removal of excess free PEG may be performed by packing a column (Tricorn Empty High-Performance Columns, GE Healthcare) with POROS 50 HQ support (Applied Biosystems), following which the column is equilibrated with equilibration buffer (25 mM Tris-HCl buffer, pH 8.2). The PEGylated AChE-R is loaded onto the equilibrated column and thereafter the column is washed with 5CV of equilibration buffer. Under these conditions, the AChE-R binds to the column PEGylated AChE-R is eluted in the next step by the elution buffer (0.3M NaCl, 25 mM Tris-HCl buffer, pH 8.2) The peak of this stage may be pooled and stored at 2-8° C. for short term, or frozen at −20° C. for long term storage.

In further still aspects, the AChE-R polypeptide may comprise a fusion protein having a first moiety, which is a AChE-R polypeptide, and a second moiety, which is a heterologous peptide or protein. Fusion proteins may include myc, HA-, or His6-tags. Fusion proteins further include the Angptl6 peptide fused to the Fc domain of a human IgG. In particular aspects, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130. The Fc moiety to can be derived from mouse IgG1 or human IgG2_{M}4. Human IgG2_{M}4 (See U.S. Published Application No. 20070148167 and U.S. Published Application No. 20060228349) is an antibody from IgG2 with mutations with which the antibody maintains normal pharmacokinetic profile but does not possess any known effector function. Fusion proteins further include the AChE-R fused to human serum albumin, transferrin, or an antibody.

In further still aspects, the AChE-R includes embodiments wherein the AChE-R is conjugated to a carrier protein such as human serum albumin, transferrin, or an antibody molecule.

The AChE variants of the present invention can be provided to the treated subject (i.e. mammal) per se (e.g., purified or directly as part of a plant) or can be provided in a pharmaceutical composition comprising the AChE-R of the present invention. As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the recombinant AChE-R of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest to edition, which is incorporated herein by reference.

Suitable peripheral routes of administration may, for example, include oral, rectal, transmucosal, especially transnasal, intestinal or parenteral delivery, including intramuscular, subcutaneous and intravenous, inrtaperitoneal, intranasal, or intraocular injections.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or to alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (nucleic acid construct) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. For example, 6-OHDA-lesioned mice may be used as animal models of Parkinson's. In addition, a sunflower test may be used to test improvement in delicate motor function by challenging the animals to open sunflowers seeds during a particular time period.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an U.S. Food and Drug Administration (FDA) approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration (FDA) for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Transgenic strains: Naïve adult mice were employed in 4 different experiments.

These included the parent strain FVB/N, TgS (transgenic mice expressing the human AChE-S variant) and TgR (transgenic mice expressing human AChE-R) mice, whose brain regions before and after exposure were used for microarray and qRT-PCR analyses. Mice were kept on a 12 h dark/12 h light diurnal schedule.

MPTP exposure (Protocol I): MPTP-HCl (Sigma, Rehovot, Israel) was dissolved in saline and injected in four doses of 20 mg/Kg at 2 hour intervals for a cumulative dose of 80 mg/Kg. 6 mice were injected from each of the three strains with saline and 6 mice with MPTP, a total of 36 mice (Protocol 1). 4 days after injection, mice were decapitated following Isoflurane (Rhodia, Bristol, UK) anaesthesia, and CPu and PFC were dissected on ice and immediately stored at −80° C. for subsequent tests.

mRNA preparation for microarray and RT-PCR analyses: RNA was extracted from frozen CPu and PFC using the RNeasy lipid tissue kit (Qiagen, Hilden, Germany) according to the manufacturer's instructions, with DNAse treatment.

Microarray procedures: Microarray procedures followed manufacturer's instructions (wwwdotaffymetrixdotcom). Affymetrix M430A 2.0 arrays were analyzed using MAS 5 software (Affymetrix) by scaling to an average intensity of 150. Each of the 12 arrays was hybridized with pooled RNA from 3-4 mice, except for one of the TgS MPTP injected groups, containing tissue from only two mice.

Differential expression analysis: Two types of analyses were conducted. In the first, a probe set was considered as changed if the Affymetrix change algorithm (wwwdotaffymetrixdotcom), showed a significant change (P<0.05) in all four possible pair wise comparisons. In the second, pairwise two-way ANOVA models compared two groups at a time and a change was considered significant at P<0.05. Further filtering was conducted for those probe sets presenting change greater than 1.5 fold. Those were done on RMA normalized samples.

Gene Ontology (GO) enrichment analysis: GO hierarchy groups were downloaded from the GO website (wwwdotgeneontologydotorg) and probe set annotations were downloaded from the Affymetrix web site (wwwdotaffymetrixdotcom). All codes for these (and other) analyses were written using the MATLAB programming language. Two complementary approaches were used on Biological Processes (BP) for this analysis. The continuous and discrete approaches for analysis of GO terms are described in detail in [Ben-Shaul, 2005 Bioinformatics 2005; 21: 1129-37].

Brain sections: For histology, brain hemispheres were dissected on ice and immediately transferred to 4% PBS-Paraformaldehyde (pH 7.4) for at least 72 hours prior to sectioning. Paraffin-embedded blocks were prepared, and coronal sections of 7 um thickness were made on SuperFrost™ Plus slides (Menzel-Glaser, Germany). Striatal sections were made in the rostral to caudal direction, starting at Bregma +1.1 coordinates.

Immunohistochemistry: Paraffin-embedded sections were re-hydrated using Xylene and serial dilutions of ethyl-alcohol in double-distilled water. Antigens were retrieved by immersing slides in citric buffer and heating in a microwave oven for 10 minutes with intermittent boiling. Sections were blocked (PBS+4% serum) and incubated overnight at room temperature with mouse anti-Splicing Factor-2 (SF2/ASF, Zymed, 32-4600, SF, USA) diluted 1/50, followed by 1 hour incubation in Cy3 conjugated goat×mouse FABs secondary antibody (Jackson, Immunoresearch), counterstained with DAPI ( 1/1000) and covered in water-based glue.

Confocal microscopy: A combination of Bio-Rad MRC-1024 confocal instrument and a Zeiss Axiovert 135M inverted microscope was used to acquire confocal images at a series of horizontal planes at intervals of 1 um. Images from all planes were then projected to yield a composite image using the ImagePro Plus software.

AChE activity: Gastrocnemius muscle, hippocampus and parietal cortex were homogenized in solution D [0.01 M Tris, 1% Triton, 1 M NaCl, 1 mM EGTA, pH 7.4], kept on ice for 1 hour, centrifuged at 14K RPM for 15 mM at 4° C., and the supernatant was collected. AChE activity was measured in plasma and tissues according to Ellman's method as detailed elsewhere [Ben-Shaul, 2006, Eur J Neurosci 2006; 23: 2915-22]. Plasma enzyme activities were normalized to μmol/min per ul and tissue AChE activity levels were normalized to nmol substrate/min*mg protein.

Protection experiments with recombinant human AChE-R (rhAChE-R): rhAChE-R was produced at Protalix Ltd. (Carmiel, Israel) in plant cell cultures from codon-optimized human AChE-R mRNA.

Single i.v. injection of Balb/C mice with 1 nmole/mouse of polyethylene glycol-encapsulated (PEGylated) rhAChE or Saline (N=9, 15, respectively) (Protocol 2) was followed by collecting whole blood samples (approximately 30 μl) into heparin-containing microhematocrit capillaries by tail sectioning. Each animal was bled 24 hours prior to dosing and at 5 of the following time points: 3, 10, 20, 40 min and 1, 2, 4, 8, 16 and 24 hours after treatment. Plasma was separated by 1000 rpm centrifugation (20 min, 4° C.) and stored at −20° C. until use. Sacrifice by Carbon dioxide asphyxiation was at 8, 16 and 24 hours post-administration. CPu, hippocampus, parietal cortex and gastrocnemius muscle were collected and immediately frozen at −80° C. until use. For prophylaxis experiments (Protocol 3), Balb/COlaHsd male mice (n=4/group) were i.v. injected with non-PEGylated rhAChE-R (50, 60, and 75 nmole/kg; 1, 1.2, and 1.5 nmole/mouse, respectively) or with PEGylated-rhAChE at 50 nmole/kg (1 nmole/mouse) to prolong the enzymes t½ in the circulation (see Evron et al.¹⁹ and Geyer et al.²⁰). Two minutes post-injection, mice were i.v. challenged with 100 nmol/Kg of DEPQ (1.33×LD₅₀) as calibrated for these mice). Control mice received saline followed by DEPQ. Toxic signs and mortality were monitored for 24 hours post-exposure.

For the PEGylated-rhAChE-R treatment experiments (Protocol 4), FVB/N female mice (n=8-12/group) were s.c. challenged with 75 μg/kg DEPQ (1.5×LD₅₀). At approximately 10, 15, or 20 minutes post-challenge, PEG-rhAChE-R at 70 or 50 nmol/Kg was i.v. administered to the tail vein. Control mice received saline approximately 1 minute following DEPQ. Toxic signs and mortality were monitored for 24 hours after challenge.

Correlation analysis, distances in spliceosomal “probe space” and the clustering factor: Correlations were calculated by considering the expression signals for all probes annotated with a specific GO biological process term (i.e. nuclear mRNA splicing) on each microarray and calculating the Pearson correlation coefficients. Distances in probe space were defined as the Euclidean distances between the signal vectors (spanning all probe sets annotated with a given functional term). For example, the distance D between the first MPTP array for FVB/N mice (FM1) and the first array for Naïve FVB/N mice (F1), is defined as D(FM1,F1)═(Σᵢ(FM1i−F1i)²)^{0.5} where the summation index i is over all probes associated with a given category. Correspondingly, the clustering factor is defined as: [D(F,S)+D(S,R)+D(R,F)]/[D(FM,SM)+D(SM,RM)+D(RM,FM)] where FM, SM, and RM denote the average values over duplicates for the MPTP treated FVB/N, TgS, and TgR groups, respectively (i.e. FM=(FM1+FM2)/2). Similarly, F, S and R denote the average values for the naïve FVB/N, TgS, and TgR groups, respectively. In geometrical terms, the clustering factor is equivalent to the ratios between the triangles depicting inter-strain distances before and after MPTP exposure.

Quantitative Real-Time PCR: 1 μg of striatal RNA was reverse transcribed using RT² First strand Kit and applied to custom-made PCR array plates (SABiosciences, Frederick, Md., USA), using ABI Prism HT-7900 sequence analyzer (Applied Biosystems, Foster City, Calif.). Data analysis was conducted with RT² Profiler PCR Array Data Analysis Software (SABiosciences; Table 1, herein below).

TABLE 1

| Symbol | Ref Seq No. | Name |
|---|---|---|
| IL4 | NM_000589 | Interleukin 4 |
| APP | NM_000484 | Amyloid beta (A4) precursor protein (peptidase nexin-II, |
| | | Alzheimer disease) |
| BACE1 | NM_138973 | Beta-site APP-cleaving enzyme 1 |
| BACE2 | NM_012105 | Beta-site APP-cleaving enzyme 2 |
| C1QA | NM_015991 | Complement component 1, q subcomponent, A chain |
| C1QB | NM_000491 | Complement component 1, q subcomponent, B chain |
| CD4 | NM_000616 | CD4 molecule |
| CD40LG | NM_000074 | CD40 ligand (TNF superfamily, member 5, hyper-IgM |
| | | syndrome) |
| CD68 | NM_001251 | CD68 molecule |
| CD8A | NM_001768 | CD8a molecule |
| CHAT | NM_020985 | Choline acetyltransferase |
| GFAP | NM_002055 | Glial fibrillary acidic protein |
| ICAM1 | NM_000201 | Intercellular adhesion molecule 1 (CD54), human rhinovirus |
| | | receptor |
| TICAM1 | NM_182919 | Toll-like receptor adaptor molecule 1 |
| IFNG | NM_000619 | Interferon, gamma |
| IFNGR1 | NM_000416 | Interferon gamma receptor 1 |
| IFNGR2 | NM_005534 | Interferon gamma receptor 2 (interferon gamma transducer 1) |
| IL10 | NM_000572 | Interleukin 10 |
| IL12A | NM_000882 | Interleukin 12A (natural killer cell stimulatory factor 1, |
| | | cytotoxic lymphocyte maturation factor 1, p35) |
| IL12B | NM_002187 | Interleukin 12B (natural killer cell stimulatory factor 2, |
| | | cytotoxic lymphocyte maturation factor 2, p40) |
| IL1A | NM_000575 | Interleukin 1, alpha |
| IL1B | NM_000576 | Interleukin 1, beta |
| IL6 | NM_000600 | Interleukin 6 (interferon, beta 2) |
| ITGAX | NM_000887 | Integrin, alpha X (complement component 3 receptor 4 |
| | | subunit) |
| MYD88 | NM_002468 | Myeloid differentiation primary response gene (88) |
| NFKB2 | NM_002502 | Nuclear factor of kappa light polypeptide gene enhancer in |
| | | B-cells 2 (p49/p100) |
| NOS1 | NM_000620 | Nitric oxide synthase 1 (neuronal) |
| PTGS1 | NM_000962 | Prostaglandin-endoperoxide synthase 1 (prostaglandin G/H |
| | | synthase and cyclooxygenase) |
| PTGS2 | NM_000963 | Prostaglandin-endoperoxide synthase 2 (prostaglandin G/H |
| | | synthase and cyclooxygenase) |
| PTPRC | NM_002838 | Protein tyrosine phosphatase, receptor type, C |
| S1OOB | NM_006272 | S100 calcium binding protein B |
| TLR2 | NM_003264 | Toll-like receptor 2 |
| TLR3 | NM_003265 | Toll-like receptor 3 |
| TLR4 | NM_138554 | Toll-like receptor 4 |
| TLR5 | NM_003268 | Toll-like receptor 5 |
| TLR7 | NM_016562 | Toll-like receptor 7 |
| TLR8 | NM_138636 | Toll-like receptor 8 |
| TLR9 | NM_017442 | Toll-like receptor 9 |
| TNF | NM_000594 | Tumor necrosis factor (TNF superfamily, member 2) |
| CD14 | | |
| Eno1 | NM_023119 | Enolase 1, alpha non-neuron |
| Eno2 | NM_013509 | Enolase 2, gamma neuronal |
| Mapk3 | NM_011952 | Mitogen-activated protein kinase 3 |
| Hsd17b10 | NM_016763 | Hydroxysteroid (17-beta) dehydrogenase 10 |
| Sfrs1 | NM_173374 | Splicing factor, arginine/serine-rich 1 (ASF/SF2) |
| Sfrs2 | NM_011358 | Splicing factor, arginine/serine-rich 2 (SC-35) |
| Pon1 | NM_011134 | Paraoxonase 1 |
| Ache | NM_009599 | Acetylcholinesterase |
| Kcnj8 | NM_008428 | Potassium inwardly-rectifying channel, subfamily J, |
| | | member 8 |
| Kcnj11 | NM_010602 | Potassium inwardly rectifying channel, subfamily J, |
| | | member 11 |
| Abcc8 | NM_011510 | ATP-binding cassette, sub-family C (CFTR/MRP), |
| | | member 8 |
| Abcc9 | NM_011511 | ATP-binding cassette, sub-family C (CFTR/MRP), |
| | | member 9 |
| Drd1a | NM_010076 | Dopamine receptor D1A |
| Drd2 | NM_010077 | Dopamine receptor 2 |
| Drd3 | NM_007877 | Dopamine receptor 3 |
| Drd4 | NM_007878 | Dopamine receptor 4 |
| Drd5 | NM_013503 | Dopamine receptor 5 |
| Maoa | NM_173740 | Monoamine oxidase A |
| Maob | NM_172778 | Monoamine oxidase B |
| Slc6a3 | NM_010020 | Solute carrier family 6 (neurotransmitter transporter, |
| | | dopamine), member 3 |
| Slc18a1 | NM_153054 | Solute carrier family 18 (vesicular monoamine), member 1 |
| Slc18a2 | NM_172523 | Solute carrier family 18 (vesicular monoamine), member 2 |
| Th | NM_009377 | Tyrosine hydroxylase |
| Comt | NM_007744 | Catechol-O-methyltransferase |
| Snca | NM_009221 | Synuclein, alpha |
| Cyp2d22 | NM_019823 | Cytochrome P450, family 2, subfamily d, polypeptide 22 |
| Gpx1 | NM_008160 | Glutathione peroxidase 1 |
| Nfe212 | NM_010902 | Nuclear factor, erythroid derived 2, like 2 |
| Sod1 | NM_011434 | Superoxide dismutase 1, soluble |
| Sod2 | NM_013671 | Superoxide dismutase 2, mitochondrial |
| Sod3 | NM_011435 | Superoxide dismutase 3, extracellular |
| Nqo1 | NM_008706 | NAD(P)H dehydrogenase, quinone 1 |
| Uchl1 | NM_011670 | Ubiquitin carboxy-terminal hydrolase L1 |
| Park2 | NM_016694 | Parkin |
| Gpr37 | NM_010338 | G protein-coupled receptor 37 |
| Adcy8 | NM_009623 | Adenylate cyclase 8 |
| Bdnf | NM_007540 | Brain derived neurotrophic factor |
| Pon3 | NM_173006 | Paraoxonase 3 |
| Nlgn1 | NM_138666 | Neuroligin 1 |
| Itgb1 | NM_010578 | Integrin beta 1 (fibronectin receptor beta) |
| Gnb2l1 | NM_008143 | Guanine nucleotide binding protein (G protein), beta |
| | | polypeptide 2 like 1 |
| Nrxn1 | NM_020252 | Neurexin I |
| Aurka | NM_011497 | Aurora kinase A |
| Aurkb | NM_011496 | Aurora kinase B |
| Aurkc | NM_020572 | Aurora kinase C |
| PPARD | NM_006238 | Peroxisome proliferator-activated receptor delta |
| PPARG | NM_015869 | Peroxisome proliferator-activated receptor gamma |
| PPARA | NM_005036 | Peroxisome proliferative activated receptor, alpha |
| PPC | SA_00103 | Positive PCR Control |
| RTC | SA_00104 | Reverse Transcription Control |
| MGDC | SA_00106 | Mouse Genomic DNA Contamination |
| Actb | NM_007393 | Actin, beta, cytoplasmic |
| Gapdh | NM_008084 | Glyceraldehyde-3-phosphate dehydrogenase |
| Gusb | NM_010368 | Glucuronidase, beta |
| Hprt1 | NM_013556 | Hypoxanthine guanine phosphoribosyl transferase 1 |
| Hsp90ab1 | NM_008302 | Heat shock protein 90kDa alpha (cytosolic), class B |
| | | member 1 |

### Example 1

### MPTP Exposure Induces Widespread Modifications in Splicing-Associated Transcripts Results

Transcriptomeal changes were first studied in the mouse PFC 72 hours following MPTP injection (Ben-Shaul et al., 2006) compared to naive FVB/N PFC. Following MPTP exposure, microarray analyses revealed massive predictable changes (Gu et al., 2003; Mandel et al., 2000; Miller et al., 2004), in numerous splicing-related to genes. Of 78 distinct UniGene (Wheeler et al., 2005) members (represented by 140 probe sets) annotated with the Gene Ontology (GO) (Ashbumer et al., 2000) term nuclear mRNA splicing, 17 (22%) included at least one probe set significantly modified according to the Affymetrix change algorithm (www.affymetrix.com.) (FIG. 1A). Table 2, herein below presents details of the probe sets shown in FIG. 1A. For each probe set, the first column shows the probe set number (i.e. the order used in FIG. 1A), followed by the gene symbol, the code used in FIG. 1A, the log ratio and the change call according to the Affymetrix criterion.

TABLE 2

| | | | | | Change |
|---|---|---|---|---|---|
| order | symbol | code | probe set | log ratio | call |
| 1 | — | 1 | 1429832_at | −0.76184 | 0 |
| 2 | — | 2 | 1431505_at | −0.09311 | 0 |
| 3 | — | 3 | 1431506_s_at | −0.00938 | 0 |
| 4 | — | 4 | 1438835_a_at | 0.36213 | 0 |
| 5 | — | 5 | 1452240_at | −0.13 | 0 |
| 6 | AI450757 | 6 | 1427134_at | 0.68031 | 1 |
| 7 | AI450757 | 6 | 1427135_at | 1.1669 | 0 |
| 8 | AI450757 | 6 | 1427136_s_at | 1.1686 | 1 |
| 9 | Brunol4 | 7 | 1426929_at | 0.61866 | 1 |
| 10 | Brunol4 | 7 | 1426930_at | 0.21188 | 0 |
| 11 | Crnkl1 | 8 | 1420849_at | 0.83591 | 0 |
| 12 | Crnkl1 | 8 | 1420850_at | 1.0918 | 1 |
| 13 | Cugbp1 | 9 | 1423932_at | 0.93922 | 0 |
| 14 | Cugbp1 | 9 | 1425932_a_at | 2.2209 | 0 |
| 15 | Cugbp1 | 9 | 1427413_a_at | 0.83076 | 0 |
| 16 | Cugbp2 | 10 | 1423895_a_at | 0.58998 | 1 |
| 17 | Cugbp2 | 10 | 1450069_a_at | 0.71333 | 1 |
| 18 | Cugbp2 | 10 | 1451154_a_at | −0.17419 | 0 |
| 19 | D18Wsu98e | 11 | 1419179_at | 0.64559 | 0 |
| 20 | Dbr1 | 12 | 1451641_at | 0.047344 | 0 |
| 21 | Ddx1 | 13 | 1415915_at | −0.24417 | 0 |
| 22 | Dhx15 | 14 | 1416144_a_at | −0.16211 | 0 |
| 23 | Dhx15 | 14 | 1416145_at | 0.058702 | 0 |
| 24 | Dhx16 | 15 | 1423925_at | 0.27389 | 0 |
| 25 | Dhx8 | 16 | 1426629_at | 0.084728 | 0 |
| 26 | Fnbp3 | 17 | 1420916_at | 0.01806 | 0 |
| 27 | Fnbp3 | 17 | 1420917_at | 1.9887 | 0 |
| 28 | Fnbp3 | 17 | 1450035_a_at | 0.65946 | 0 |
| 29 | Gemin6 | 18 | 1424300_at | −0.42497 | 0 |
| 30 | Gemin7 | 19 | 1435764_a_at | −0.14272 | 0 |
| 31 | Lsm1 | 20 | 1423873_at | −0.23961 | 0 |
| 32 | Lsm3 | 21 | 1448536_at | 0.18294 | 0 |
| 33 | Lsm4 | 22 | 1448622_at | −0.09676 | 0 |
| 34 | Lsm8 | 23 | 1448703_at | −0.0255 | 0 |
| 35 | Nol3 | 24 | 1451503_at | −0.12997 | 0 |
| 36 | Nono | 25 | 1415820_x_at | −0.16505 | 0 |
| 37 | Nono | 25 | 1431239_at | 1.8272 | 0 |
| 38 | Nono | 25 | 1448103_s_at | −0.08254 | 0 |
| 39 | Nova1 | 26 | 1426938_at | −0.27571 | 0 |
| 40 | Nova1 | 26 | 1452245_at | 0.50779 | 0 |
| 41 | Phf5a | 27 | 1424170_at | 0.10372 | 0 |
| 42 | Plrg1 | 28 | 1448282_at | −0.08303 | 0 |
| 43 | Prpf3 | 29 | 1424314_at | 0.54073 | 0 |
| 44 | Prpf4b | 30 | 1425497_a_at | 1.0979 | 1 |
| 45 | Prpf4b | 30 | 1425498_at | −0.08125 | 0 |
| 46 | Prpf4b | 30 | 1436427_at | 1.1301 | 1 |
| 47 | Prpf4b | 30 | 1451732_at | 1.7047 | 0 |
| 48 | Prpf4b | 30 | 1451909_a_at | 1.1706 | 1 |
| 49 | Prpf4b | 30 | 1455696_a_at | 0.7041 | 1 |
| 50 | Prpf8 | 31 | 1422453_at | −0.32208 | 0 |
| 51 | Ptbp1 | 32 | 1424874_a_at | 0.15314 | 0 |
| 52 | Ptbp1 | 32 | 1450443_at | −0.68871 | 0 |
| 53 | Ptbp2 | 33 | 1423470_at | 0.036566 | 0 |
| 54 | Ptbp2 | 33 | 1423471_at | −0.18252 | 0 |
| 55 | Rbm17 | 34 | 1452691_at | −0.05414 | 0 |
| 56 | Rbm8 | 35 | 1418119_at | −0.16993 | 0 |
| 57 | Rbm8 | 35 | 1418120_at | 0.12052 | 0 |
| 58 | Refbp1 | 36 | 1417724_at | −0.11325 | 0 |
| 59 | Refbp2 | 37 | 1422993_s_at | −0.20118 | 0 |
| 60 | Rnpc2 | 38 | 1420982_at | 0.18334 | 0 |
| 61 | Rnpc2 | 38 | 1426671_a_at | 0.13555 | 0 |
| 62 | Rnpc2 | 38 | 1438397_a_at | 1.4237 | 1 |
| 63 | Rnpc2 | 38 | 1438398_at | 2.5502 | 0 |
| 64 | Rnpc2 | 38 | 1456386_at | 1.5604 | 1 |
| 65 | Sf3a1 | 39 | 1419087_s_at | 0.18922 | 0 |
| 66 | Sf3a1 | 39 | 1449333_at | −0.20667 | 0 |
| 67 | Sf3a2 | 40 | 1450576_a_at | −0.21962 | 0 |
| 68 | Sf3a2 | 40 | 1455546_s_at | −0.1281 | 0 |
| 69 | Sf3a3 | 41 | 1423811_at | −0.10956 | 0 |
| 70 | Sf3a3 | 41 | 1432488_a_at | 0.52679 | 0 |
| 71 | Sf3b1 | 42 | 1418561_at | 0.15517 | 0 |
| 72 | Sf3b1 | 42 | 1418562_at | 1.7838 | 1 |
| 73 | Sf3b1 | 42 | 1449138_at | 0.6179 | 1 |
| 74 | Sf3b4 | 43 | 1424619_at | −0.28477 | 0 |
| 75 | Sf3b4 | 43 | 1436784_x_at | −0.26295 | 0 |
| 76 | Sfrs1 | 44 | 1428099_a_at | 0.128 | 0 |
| 77 | Sfrs1 | 44 | 1430982_at | 0.21922 | 0 |
| 78 | Sfrs1 | 44 | 1434972_x_at | 0.33836 | 0 |
| 79 | Sfrs1 | 44 | 1452430_s_at | 0.86697 | 1 |
| 80 | Sfrs1 | 44 | 1453722_s_at | 0.20247 | 0 |
| 81 | Sfrs16 | 45 | 1428961_a_at | 0.54382 | 1 |
| 82 | Sfrs2 | 46 | 1415807_s_at | 0.41546 | 1 |
| 83 | Sfrs2 | 46 | 1427504_s_at | 0.049836 | 0 |
| 84 | Sfrs2 | 46 | 1427815_at | −0.70866 | 0 |
| 85 | Sfrs2 | 46 | 1427816_at | −0.57891 | 0 |
| 86 | Sfrs2 | 46 | 1452439_s_at | 0.48085 | 1 |
| 87 | Sfrs3 | 47 | 1416150_a_at | 0.36111 | 0 |
| 88 | Sfrs3 | 47 | 1416151_at | −0.2157 | 0 |
| 89 | Sfrs3 | 47 | 1416152_a_at | −0.00805 | 0 |
| 90 | Sfrs3 | 47 | 1434512_x_at | −0.21089 | 0 |
| 91 | Sfrs3 | 47 | 1438215_at | 1.4179 | 1 |
| 92 | Sfrs3 | 47 | 1454993_a_at | −0.16567 | 0 |
| 93 | Sfrs4 | 48 | 1448778_at | −0.02822 | 0 |
| 94 | Sfrs5 | 49 | 1423130_a_at | 1.1579 | 1 |
| 95 | Sfrs6 | 50 | 1416720_at | 0.5382 | 1 |
| 96 | Sfrs6 | 50 | 1416721_s_at | 0.5728 | 1 |
| 97 | Sfrs6 | 50 | 1448454_at | 0.85509 | 1 |
| 98 | Sfrs7 | 51 | 1424033_at | 0.4078 | 0 |
| 99 | Sfrs7 | 51 | 1424883_s_at | 0.49655 | 0 |
| 100 | Sfrs7 | 51 | 1436871_at | 1.2227 | 0 |
| 101 | Sfrs9 | 52 | 1417727_at | 0.13245 | 0 |
| 102 | Snrp116 | 53 | 1416557_a_at | 0.11302 | 0 |
| 103 | Snrp116 | 53 | 1456107_x_at | 0.09229 | 0 |
| 104 | Snrpd1 | 54 | 1416336_s_at | −0.53426 | −1 |
| 105 | Snrpd2 | 55 | 1452680_at | −0.23108 | 0 |
| 106 | Snrpg | 56 | 1448357_at | −0.3639 | 0 |
| 107 | Snrpg | 56 | 1448358_s_at | −0.31889 | 0 |
| 108 | Srrm1 | 57 | 1420934_a_at | 0.31471 | 1 |
| 109 | Srrm1 | 57 | 1420935_a_at | 2.3486 | 0 |
| 110 | Srrm1 | 57 | 1450045_at | −0.72186 | 0 |
| 111 | Srrm1 | 57 | 1454689_at | 0.55832 | 1 |
| 112 | Syncrip | 58 | 1422768_at | −0.29675 | 0 |
| 113 | Syncrip | 58 | 1422769_at | 0.71469 | 1 |
| 114 | Syncrip | 58 | 1426402_at | −0.13733 | 0 |
| 115 | Syncrip | 58 | 1450743_s_at | 0.72775 | 1 |
| 116 | Thoc1 | 59 | 1424641_a_at | 0.70052 | 1 |
| 117 | Thoc1 | 59 | 1424642_at | 1.0818 | 1 |
| 118 | Thoc1 | 59 | 1437714_x_at | −0.32077 | 0 |
| 119 | Thoc1 | 59 | 1455829_at | −0.25934 | 0 |
| 120 | U2af1 | 60 | 1422509_at | 0.11623 | 0 |
| 121 | U2af2 | 61 | 1417260_at | −0.07775 | 0 |
| 122 | Zfp162 | 62 | 1422321_a_at | 0.10001 | 0 |
| 123 | Zfp162 | 62 | 1423750_a_at | −0.32947 | 0 |
| 124 | Zfp162 | 62 | 1423751_at | −0.31232 | 0 |
| 125 | 6330548N22Rik | 63 | 1424554_at | −0.20038 | 0 |
| 126 | 6330437E22Rik | 64 | 1438674_a_at | 1.7151 | 1 |
| 127 | 6330437E22Rik | 65 | 1438675_at | 1.8188 | 0 |
| 128 | 2610031L17Rik | 66 | 1424036_at | −0.07762 | 0 |
| 129 | 2610031L17Rik | 67 | 1426026_at | −0.30307 | 0 |
| 130 | 2610031L17Rik | 68 | 1454789_x_at | −0.42471 | 0 |
| 131 | 2410044K02Rik | 69 | 1423970_at | −0.15984 | 0 |
| 132 | 2410044K02Rik | 70 | 1423971_at | −0.51301 | 0 |
| 133 | 6330548N22Rik | 71 | 1435821_s_at | −0.12611 | 0 |
| 134 | 0610009D07Rik | 72 | 1417054_a_at | −0.36221 | 0 |
| 135 | 0610009D07Rik | 73 | 1417055_at | 0.21847 | 0 |
| 136 | 0610009D07Rik | 74 | 1435508_x_at | −0.98313 | 0 |
| 137 | 0610009D07Rik | 75 | 1436681_x_at | −0.33523 | 0 |
| 138 | 2010003O18Rik | 76 | 1425211_at | −0.16942 | 0 |
| 139 | 1100001J08Rik | 77 | 1424136_a_at | −0.57696 | 0 |

Increases were observed in sfrs 1 (ASF/SF2), sfrs2 (SC35), sfrs3 (SRp20), sfrs5 (SRp40), sfrs6, 7 and 16 and sf3b. Srpk2 and clk4, two prominent splicing factor protein kinases, which affect the activity and cellular localization of splicing factors (Black, 2003) were also increased. A correlation matrix of these expression patterns of the 140 splicing-related genes for pooled brains from naive and MPTP exposed mice (FIG. 1B) showed within-treatment reproducibility, and that MPTP markedly alters these splicing-related genes.

Next, the scope of detected changes were compared in individual splicing-related transcripts (wwwdotaffymetrixdotcom) to the expectation given the entire set of to measured transcripts (FIG. 1C). This involved the discrete approach (Ben-Shaul et al., 2005), testing whether the number of changed (increased or decreased) transcripts annotated with a given GO term exceeds that expected by chance and continuous analysis of the distribution of changes for all genes associated with a given term. Both methods identified changes in PD-affected processes (e.g. dopamine synthesis, oxygen and Reactive Oxygen Species metabolism, inflammatory response and ubiquitin cycle. Increases were also detected by both approaches in splice site selection (P<0.05, FIG. 1C). Both the number of changed transcripts and the category as a whole were accentuated under MPTP exposure. For example, serine-arginine rich (SR) proteins (Meshorer et al., 2005b), (Long and Caceres, 2009) were modified, and nuclear mRNA splicing transcripts (a grandparent term of splice site selection) revealed a trend (p=0.052) according to the continuous approach (FIGS. 1A-C). Linking the two terms, changes in spliceosome assembly were detected by the discrete method alone, reflecting significant change in a few members of this subgroup.

### Example 2

### AChE Variant Strains Show Different Reactions to MPTP Exposure

To find out if the observed differences induce secondary changes effecting brain function, transgenic strains with enforced expression of human AChE-S or AChE-R (TgS, TgR) (Shaked et al., 2009; Sternfeld et al., 2000) were selected. The PFC and CPu transcriptomes of naïve TgS mice showed more changed genes than TgR as compared to FVB/N mice. In both strains, the CPu exhibited more changes than in the PFC (FIG. 2A). In contrast, exposure to MPTP (FIG. 2B) induced many more differential expression changes in the PFC than in the CPu, again more profoundly in TgS mice. For validation tests, 78 inflammation and signaling-related transcripts were subjected to real-time PCR. Of those, 65 (i.e. 83%) revealed consistent directions of change in the microarrays and qRT-PCR for all comparisons made (Naïve or MPTP-exposed TgS and TgR vs. FVB/N mice). More changes occurred in MPTP-exposed TgR than FVB/N mice (e.g. in Toll-Like receptors 4 and 9 (TLR 4 and TLR9), monoamine oxidase A (MAO A) and Dopamine receptor 2 (Drd2) ((FIG. 2C)).

### Example 3

### MPTP Exposure Induces Concerted Shifts in the Spliceosomal Configuration

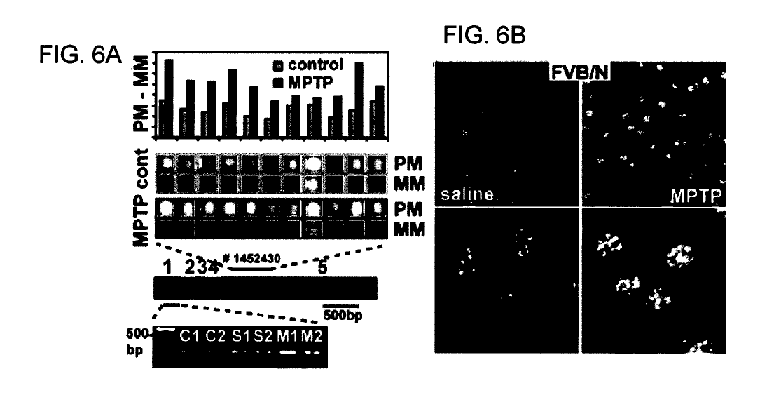

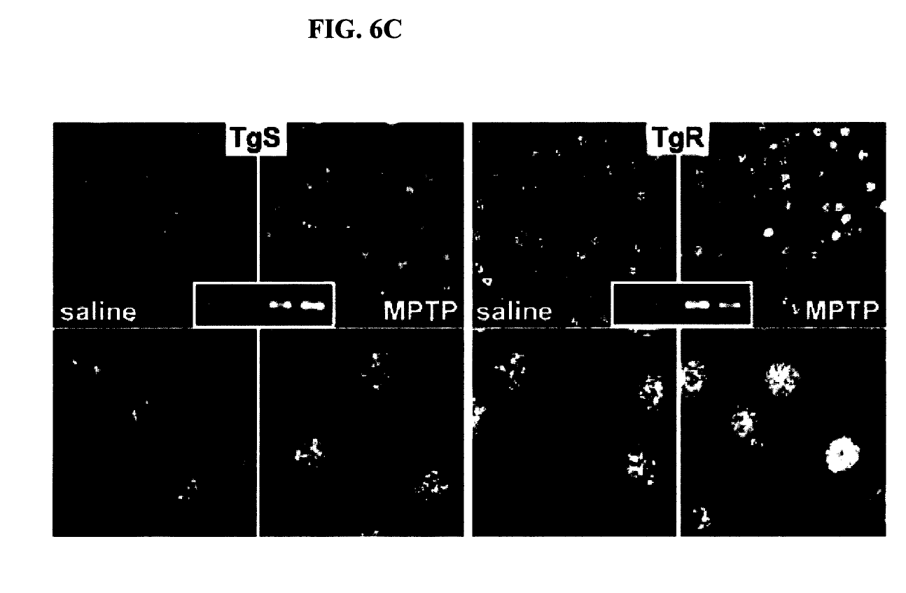

The expression levels of selected splicing-related transcripts (e.g. ASF/SF2, SC35, Cugbp2, Snrpd2, Prpf4b, Crnkl1, FIG. 3A) from different strains were distinct in naïve mice yet clustered together following MPTP exposure (FIGS. 3B-C). TgR mice further presented larger, and TgS— smaller MPTP-induced increments than FVB/N controls in nuclear ASF/SF2 labeling (FIGS. 6A-C). Between all possible pairs of the 12 arrays (2 biological pools×3 strains×2 treatments), differences in splicing-regulating transcripts were observed as well as in other transcript classes.

Following MPTP exposure, the expression patterns of alternative splicing transcripts attained highly similar configurations across all strains (FIGS. 3B-C). Euclidian distances in the 140-dimensional space (defined by expression levels of splicing-related probes) for pairs of strain×treatment combinations demonstrated initial between-strain distances which were markedly reduced under MPTP (FIG. 3D). Of note, distances in the spliceosomal space are also affected by (normalized) absolute expression levels, unlike the correlation analysis (FIGS. 3B-C), which is sensitive only to the relative expression patterns of transcripts. These distances thus demonstrate the summed efficacy of the induced transcription and splicing changes. To quantify the extent of MPTP-induced similarity in these expression patterns, we have further defined the “clustering factor” as the ratio of between-strain distances before and after MPTP exposure (presented as the ratio between the R—F—S and the RM-FM-FS triangles in FIG. 3D). The clustering factor for the path of GO biological process terms revealed increases as the hierarchy descends along this path (FIG. 3E), especially for probes associated with splice site selection.

### Example 4

### Role of AChE-R in Body to Brain Interactions

To determine if body-to-brain signaling was involved, peripheral AChE-R levels were elevated. Injection of highly purified recombinant human PEGylated AChE-R (rhAChE-R) produced in plant cell cultures (i.v., lnmole/mouse) raised plasma AChE activity by 946-fold within 3 minutes, with circulatory t½ of 40 minutes (FIG. 4A). Saline-injected mice showed no significant change in AChE activity, excluding stress effects as a factor (Kaufer et al., 1998). In the gastrocnemius muscle, AChE activity in the rhAChE-R injected group increased by 67% by 8 hours post-injection as compared to to controls (3.5±0.06 vs. 2.1±0.2 nmole/min*mg, respectively, p=0.006, Student's t test) (FIG. 4B). By 24 hours post-injection, muscle AChE activity returned to baseline. In the parietal cortex and hippocampus, AChE activity remained essentially unchanged (FIGS. 4C-D).

### Example 5

### Peripherally Administrated rhAChE-R Induces Changes in CPu Gene Expression

By 8 and 16 hours post-injection, the Parkinsonian CPu of mice peripherally injected with rhAChE-R showed profound increases in 23 and 12 out of the 88 tested transcripts (similar to those in FIGS. 2C-F; Tables 3A and 3B). Interleukin 1α and β and Toll-like receptors 4 and 7 were elevated, likely reflecting enhanced neuro-immune response in injected animals. Also, the PD-related D3 dopamine receptor, the antioxidant superoxide dismutase 2 (SOD2) and the E3 ubiquitin ligase Parkin were over-expressed.

Table 3A lists the 23 genes which were up-regulated in the CPu of rhAChE-R as compared to saline-injected mice, 8 hr post-injection. Table 3B lists the genes which were regulated in the CPu of rhAChE-R as compared to saline-injected mice at 16 hr post-injection, 11 genes were up-regulated by rhAChE-R injection, and one was down-regulated.

TABLE 3A

| Gene | | Fold | P- |
|---|---|---|---|
| Symbol | Gene name | Regulation | value |
| IL1B | Interleukin 1 beta | 3.11 | 0.0016 |
| IL1A | Interleukin 1 alpha | 2.58 | 0.0003 |
| Sod2 | Superoxide dismutase 2, | 1.83 | 0.035 |
| | mitochondrial | | |
| IL4 | Interleukin 4 | 1.64 | 0.028 |
| Aurka | Aurora kinase A | 1.58 | 0.021 |
| ICAM1 | Intercellular adhesion molecule 1 | 1.56 | 0.002 |
| Drd3 | Dopamine receptor 3 | 1.53 | 0.020 |
| C1QB | Complement component 1, q | 1.52 | 0.001 |
| | subcomponent, beta polypeptide | | |
| Park2 | Parkin | 1.42 | 0.006 |
| Pon3 | Paraoxonase 3 | 1.42 | 0.008 |
| Slc18a2 | Solute carrier family 18 (vesicular | 1.39 | 0.031 |
| | monoamine), member 2 | | |
| IL10 | Interleukin 10 | 1.37 | 0.037 |
| Comt | Catechol-O-methyltransferase | 1.35 | 0.012 |
| CD14 | CD14 antigen | 1.34 | 0.002 |
| TLR9 | Toll-like receptor 9 | 1.28 | 0.039 |
| NOS1 | Nitric oxide synthase 1, neuronal | 1.27 | 0.008 |
| Kcnj11 | Potassium inwardly rectifying | 1.24 | 0.035 |
| | channel, subfamily J, member 11 | | |
| Ache | Acetylcholinesterase | 1.23 | 0.044 |
| Eno2 | Enolase 2, gamma neuronal | 1.23 | 0.033 |
| Nlgn1 | Neuroligin 1 | 1.22 | 0.006 |
| C1QA | Complement component 1, q | 1.20 | 0.028 |
| | subcomponent, alpha polypeptide | | |
| Gpx1 | Glutathione peroxidase 1 | 1.17 | 0.037 |
| Maob | Monoamine oxidase B | 1.16 | 0.011 |

TABLE 3B

| Gene | | Fold | P- |
|---|---|---|---|
| Symbol | Gene name | Regulation | value |
| PTPRC | Protein tyrosine phosphatase | 2.10 | 0.039 |
| | receptor type C | | |
| Uchl1 | Ubiquitin carboxy-terminal | 1.98 | 0.001 |
| | hydrolase L1 | | |
| TLR4 | Toll-like receptor 4 | 1.78 | 0.001 |
| Aurkc | Aurora kinase C | 1.59 | 0.023 |
| TLR7 | Toll-like receptor 7 | 1.53 | 0.015 |
| Drd4 | Dopamine receptor 4 | 1.46 | 0.013 |
| Pon3 | Paraoxonase 3 | 1.26 | 0.012 |
| Park2 | Parkin | 1.20 | 0.011 |
| CD68 | CD68 antigen | 1.19 | 0.029 |
| C1QA | Complement component 1, q | 1.18 | 0.007 |
| | subcomponent, alpha | | |
| | polypeptide | | |
| Maoa | Monoamine oxidase A | 1.15 | 0.024 |
| Sod3 | Superoxide dismutase 3, | −1.19 | 0.028 |
| | extracellular | | |

### Example 6

### AChE Variant Strains Show Different Reactions to MPTP Exposure

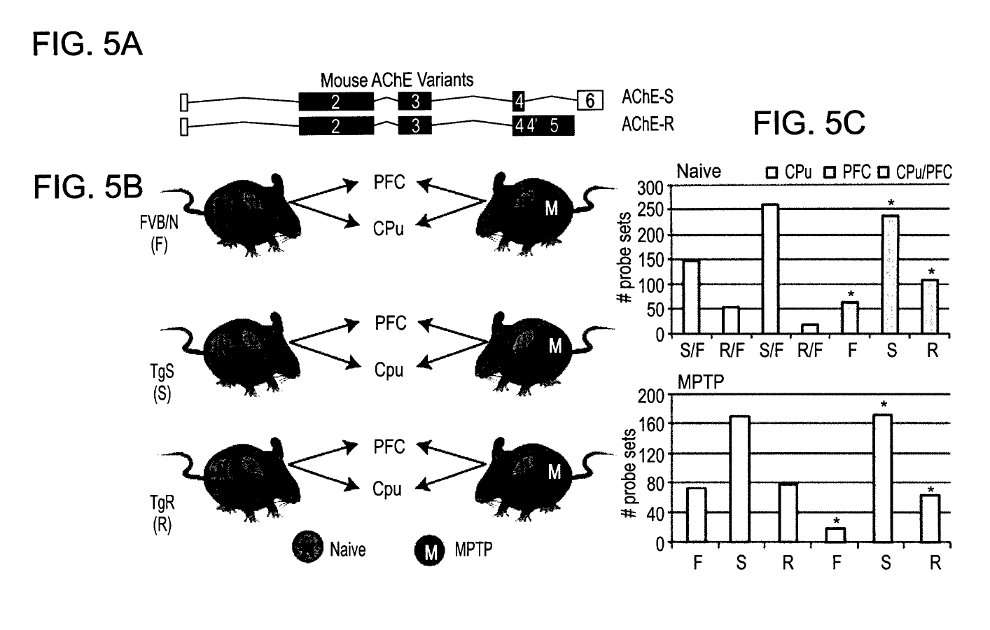

The baseline expression differences among these TgR, TgS and FVB/N mice were studied in comparison to wild-type FVB/N mice, and then their responses to the MPTP challenge were compared relative to the baseline state of each strain. Of note, both transgenic strains also express the mouse host transcripts (FIG. 5A). Using comparative ANOVA models (Inc), gene expression changes were studied in the CPu and PFC of TgR and TgS mice (FIGS. 5B-C). Intriguingly, CPu and PFC from naive to TgR presented only mildly modified transcriptome profiles as compared to controls (FIG. 5D). In contrast, TgS mice showed profound changes in both brain areas, judging from both the number of genes and from the magnitude of change in their expression levels compared to FVB/N mice (FIG. 5D). In each strain, the expression profiles of CPu compared to the PFC showed differential brain-region dependent expression patterns (FIG. 5D). Following exposure to MPTP, the same trend was evident i.e both the TgS CPu and PFC showed the highest number of transcripts changed judging by the number of changed probe sets (>160, FIG. 5D). An in-depth inspection of the SR protein ASF/SF2 (sfrs1), revealed that changes at the mRNA level were accompanied by changes in protein abundance and nuclear distribution, suggesting that these changes are physiologically relevant (FIGS. 6A-B) (Yeakley et al., 1999). Post-exposure TgR nuclei showed more prominent changes in ASF/SF2 expression, whereas TgS nuclei showed less pronounced changes than strain-matched FVB/N controls. Of note, ASF/SF2 mRNA showed changes reminiscent of those observed for the sub-nuclear distribution patterns (FIG. 6C) of the ASF/SF2 protein in all of the tested strains.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. Xu Q, Modrek B, Lee C. Genome-wide detection of tissue-specific alternative splicing in the human transcriptome. Nucleic Acids Res 2002; 30(17): 3754-3766.
2. Stamm S, Ben-Ari S, Rafalska I, Tang Y, Zhang Z, Toiber D et al. Function of alternative splicing. Gene 2005; 344: 1-20.
3. Schwerk C, Schulze-Osthoff K. Regulation of apoptosis by alternative pre-mRNA splicing. Mol Cell 2005; 19(1): 1-13.
4. Smith M J, Sharples R A, Evin G, McLean C A, Dean B, Pavey G et al. Expression of truncated presenilin 2 splice variant in Alzheimer's disease, bipolar disorder, and schizophrenia brain cortex. Brain Res Mol Brain Res 2004; 127(1-2): 128-135.
5. Jiang Z, Cote J, Kwon J M, Goate A M, Wu J Y. Aberrant splicing of tau pre-mRNA caused by intronic mutations associated with the inherited dementia frontotemporal dementia with parkinsonism linked to chromosome 17. Mol Cell Biol 2000; 20(11): 4036-4048.
6. West A, Periquet M, Lincoln S, Lucking C B, Nicholl D, Bonifati V et al. Complex relationship between Parkin mutations and Parkinson disease. Am J Med Genet 2002; 114(5): 584-591.
7. Berke J D, Sgambato V, Zhu P P, Lavoie B, Vincent M, Krause M et al. Dopamine and glutamate induce distinct striatal splice forms of Ania-6, an RNA polymerase II-associated cyclin. Neuron 2001; 32(2): 277-287.
8. Zhou F M, Wilson C, Dani J A. Muscarinic and nicotinic cholinergic mechanisms in the mesostriatal dopamine systems. Neuroscientist 2003; 9(1): 23-36.
9. Benmoyal-Segal L, Soreq H. Gene-environment interactions in sporadic Parkinson's disease. J Neurochem 2006; 97(6): 1740-1755.
10. Manthripragada A D, Costello S, Cockburn M G, Bronstein J M, Ritz B. Paraoxonase 1, agricultural organophosphate exposure, and Parkinson disease. Epidemiology 2010; 21(1): 87-94.
11. Friedman A, Kaufer D, Shemer J, Hendler I, Soreq H, Tur-Kaspa I. Pyridostigmine brain penetration under stress enhances neuronal excitability and induces early immediate transcriptional response. Nat Med 1996; 2(12): 1382-1385.
12. Kaufer D, Friedman A, Seidman S, Soreq H. Acute stress facilitates long-lasting changes in cholinergic gene expression. Nature 1998; 393(6683): 373-377.
13. Benmoyal-Segal L, Vander T, Shifman S, Bryk B, Ebstein R P, Marcus E L et al. Acetylcholinesterase/paraoxonase interactions increase the risk of insecticide-induced Parkinson's disease. Faseb J 2005; 19(3): 452-454.
14. Ben-Shaul Y, Benmoyal-Segal L, Ben-Ari S, Bergman H, Soreq H. Adaptive acetylcholinesterase splicing patterns attenuate 1-methyl-4-phenyl-1,2,3,6-tetrahydropyridine-induced Parkinsonism in mice. Eur J Neurosci 2006; 23(11): 2915-2922.
15. Dauer W, Przedborski S. Parkinson's disease: mechanisms and models. Neuron 2003; 39(6): 889-909.
16. Dery K J, Gusti V, Gaur S, Shively J E, Yen Y, Gaur R K. Alternative splicing as a therapeutic target for human diseases. Methods Mol Biol 2009; 555: 127-144.
17. Ben-Shaul Y, Bergman H, Soreq H. Identifying subtle interrelated changes in functional gene categories using continuous measures of gene expression. Bioinformatics 2005; 21(7): 1129-1137.
18. Meshorer E, Biton I E, Ben-Shaul Y, Ben-Ari S, Assaf Y, Soreq H et al. Chronic cholinergic imbalances promote brain diffusion and transport abnormalities. Faseb J 2005; 19(8): 910-922.
19. Evron T, Geyer B C, Chemi I, Muralidharan M, Kilbourne J, Fletcher S P et al. Plant-derived human acetylcholinesterase-R provides protection from lethal organophosphate poisoning and its chronic aftermath. Faseb J 2007; 21(11): 2961-2969.
20. Geyer B C, Woods R R, Mor T S. Increased organophosphate scavenging in a butyrylcholinesterase mutant. Chem Biol Interact 2008; 175(1-3): 376-379.
21. Gu G, Deutch A Y, Franklin J, Levy S, Wallace D C, Zhang J. Profiling genes related to mitochondrial function in mice treated with N-methyl-4-phenyl-1,2,3,6-tetrahydropyridine. Biochem Biophys Res Commun 2003; 308(1): 197-205.
22. Mandel S, Grunblatt E, Youdim M. cDNA microarray to study gene expression of dopaminergic neurodegeneration and neuroprotection in MPTP and 6-hydroxydopamine models: implications for idiopathic Parkinson's disease. J Neural Transm Suppl 2000; (60): 117-124.
23. Miller R M, Callahan L M, Casaceli C, Chen L, Kiser G L, Chui B et al. Dysregulation of gene expression in the 1-methyl-4-phenyl-1,2,3,6-tetrahydropyridine-lesioned mouse substantia nigra. J Neurosci 2004; 24(34): 7445-7454.
24. Wheeler D L, Barrett T, Benson D A, Bryant S H, Canese K, Church D M et al. Database resources of the National Center for Biotechnology Information. Nucleic Acids Res 2005; 33(Database issue): D39-45.
25. Ashburner M, Ball C A, Blake J A, Botstein D, Butler H, Chemy J M et al. Gene ontology: tool for the unification of biology. The Gene Ontology Consortium. Nat Genet 2000; 25(1): 25-29.
26. wwwdotaffymetrixdotcom.
27. Black D L. Mechanisms of alternative pre-messenger RNA splicing. Annu Rev Biochem 2003; 72: 291-336.
28. Meshorer E, Bryk B, Toiber D, Cohen J, Podoly E, Dori A et al. SC35 promotes sustainable stress-induced alternative splicing of neuronal acetylcholinesterase mRNA. Mol Psychiatry 2005; 10(11): 985-997.
29. Long J C, Caceres J F. The SR protein family of splicing factors: master regulators of gene expression. Biochem J 2009; 417(1): 15-27.
30. Shaked I, Meerson A, Wolf Y, Avni R, Greenberg D, Gilboa-Geffen A et al. MicroRNA-132 potentiates cholinergic anti-inflammatory signaling by targeting acetylcholinesterase. Immunity 2009; 31(6): 965-973.
31. Sternfeld M, Shoham S, Klein O, Flores-Flores C, Evron T, Idelson G H et al. Excess “read-through” acetylcholinesterase attenuates but the “synaptic” variant intensifies neurodeterioration correlates. Proc Natl Acad Sci USA 2000; 97(15): 8647-8652.
32. Fuchs J, Tichopad A, Golub Y, Munz M, Schweitzer K J, Wolf B et al. Genetic variability in the SNCA gene influences alpha-synuclein levels in the blood and brain. Faseb J 2008; 22(5): 1327-1334.
33. D'Souza I, Schellenberg G D. Regulation of tau isoform expression and dementia. Biochim Biophys Acta 2005; 1739(2-3): 104-115.
34. Hatano T, Kubo S, Sato S, Hattori N. Pathogenesis of familial Parkinson's disease: new insights based on monogenic forms of Parkinson's disease. J Neurochem 2009; 111(5): 1075-1093.
35. Elbaz A, Clavel J, Rathouz P J, Moisan F, Galanaud J P, Delemotte B et al. Professional exposure to pesticides and Parkinson disease. Ann Neurol 2009; 66(4): 494-504.
36. Metz G A. Stress as a modulator of motor system function and pathology. Rev Neurosci 2007; 18(3-4): 209-222.
37. Goldman S M, Tanner C M, Oakes D, Bhudhikanok G S, Gupta A, Langston J W. Head injury and Parkinson's disease risk in twins. Ann Neurol 2006; 60(1): 65-72.
38. Lee J K, Tran T, Tansey M G. Neuroinflammation in Parkinson's disease. J Neuroimmune Pharmacol 2009; 4(4): 419-429.
39. Darreh-Shori T, Hellstrom-Lindahl E, Flores-Flores C, Guan Z Z, Soreq H, Nordberg A. Long-lasting acetylcholinesterase splice variations in anticholinesterase-treated Alzheimer's disease patients. J Neurochem 2004; 88(5): 1102-1113.
40. Berson A, Knobloch M, Hanan M, Diamant S, Sharoni M, Schuppli D et al. Changes in readthrough acetylcholinesterase expression modulate amyloid-beta pathology. Brain 2008; 131(Pt 1): 109-119.
41. Opalinska J B, Gewirtz A M. Nucleic-acid therapeutics: basic principles and recent applications. Nat Rev Drug Discov 2002; 1(7): 503-514.
42. Sun S, Zhang Z, Sinha R, Karni R, Krainer A R. SF2/ASF autoregulation involves multiple layers of post-transcriptional and translational control. Nat Struct Mol Biol 2010.
43. Bove J, Prou D, Perier C, Przedborski S. Toxin-induced models of Parkinson's disease. NeuroRx 2005; 2(3): 484-494.
44. Rosin D L, Clark W A, Goldstein M, Roth R H, Deutch A Y. Effects of 6-hydroxydopamine lesions of the prefrontal cortex on tyrosine hydroxylase activity in mesolimbic and nigrostriatal dopamine systems. Neuroscience 1992; 48(4): 831-839.
45. McGeorge A J, Fault R L. The organization of the projection from the cerebral cortex to the striatum in the rat. Neuroscience 1989; 29(3): 503-537.
46. Kim J M, Lee K H, Jeon Y J, Oh J H, Jeong S Y, Song I S et al. Identification of genes related to Parkinson's disease using expressed sequence tags. DNA Res 2006; 13(6): 275-286.
47. Soreq L, Israel Z, Bergman H, Soreq H. Advanced microarray analysis highlights modified neuro-immune signaling in nucleated blood cells from Parkinson's disease patients. J Neuroimmunol 2008; 201-202: 227-236.
48. Blalock J E, Smith E M. Conceptual development of the immune system as a sixth sense. Brain Behav Immun 2007; 21(1): 23-33.
49. Wyss-Coray T, Mucke L. Inflammation in neurodegenerative disease—a double-edged sword. Neuron 2002; 35(3): 419-432.
50. Hickey W F. Basic principles of immunological surveillance of the normal central nervous system. Glia 2001; 36(2): 118-124.
51. Pavlov V A, Tracey K J. Controlling inflammation: the cholinergic anti-inflammatory pathway. Biochem Soc Trans 2006; 34(Pt 6): 1037-1040.
52. Tracey K J. Physiology and immunology of the cholinergic antiinflammatory pathway. J Clin Invest 2007; 117(2): 289-296.
53. Bajgar J, Fusek J, Kassa J, Kuca K, Jun D. Chemical aspects of pharmacological prophylaxis against nerve agent poisoning. Curr Med Chem 2009; 16(23): 2977-2986.
54. Raveh L, Grunwald J, Marcus D, Papier Y, Cohen E, Ashani Y. Human butyrylcholinesterase as a general prophylactic antidote for nerve agent toxicity. In vitro and in vivo quantitative characterization. Biochem Pharmacol 1993; 45(12): 2465-2474.
55. Inc P, 2008. Partek® Discovery Suite™ Partek Inc., St. Louis.

1. A method of treating or preventing Parkinson's disease in a subject in need thereof, the method comprising administering to the subject a therapeutically effective amount of an acetylcholine esterase-R (AChE-R) polypeptide, wherein the AChE-R polypeptide is devoid of an N-terminal extension to thereby treat the Parkinson's disease in the subject.

2. The method of claim 1, wherein said AChE-R polypeptide comprises a recombinant AChE-R polypeptide.

3. The method of claim 2, wherein said recombinant AChE-R is plant produced AChE-R.

4. The method of claim 1, wherein said AChE-R polypeptide has an amino acid sequence as set forth in SEQ ID NOs. 1 and 3.

5. The method of claim 1, wherein said administering is peripherally administering.

6. A method of treating or preventing Parkinson's disease in a subject in need thereof, the method comprising administering to the subject a therapeutically effective amount of an ACNE-R polypeptide, wherein the AChE-R polypeptide comprises a modification for increasing bioavailability, thereby treating the Parkinson's disease in the subject.

7. The method of claim 6, wherein said AChE-R polypeptide is devoid of an N-terminal extension.

8. The method of claim 6, wherein said AChE-R polypeptide comprises an N-terminal extension.

9. The method of claim 8, wherein said N-terminal extension is at least 90% homologous to SEQ ID NO: 2.

10. The method of claim 6, wherein said AChE-R polypeptide comprises recombinant AChE-R.

11. The method of claim 10, wherein said recombinant AChE-R is plant produced AChE-R.

12. The method of claim 6, wherein said AChE-R polypeptide has an amino acid sequences as set forth in SEQ ID NOs. 1 and 3.

13. The method of claim 6, wherein said modification comprises attachment to a heterologous polypeptide.

14. The method of claim 13, wherein said heterologous polypeptide is selected from the group consisting of human serum albumin, immunoglobulin, and transferrin.

15. The method of claim 14, wherein said immunoglobulin comprises an Fc domain.

16. The method of claim 6, wherein said modification comprises attachment to a polymer.

17. The method of claim 16, wherein said polymer is selected from the group consisting of a polycationic polymer, a non-ionic water-soluble polymer, a polyether polymer and a biocompatible polymer.

18. The method of claim 16, wherein said polymer is poly(ethylene glycol).

19. The method of claim 6, wherein said administering is peripherally administering.
